(19) United States
(12) Patent Application Publication
(10) Pub. No.: US 2024/0081657 A1
(43) Pub. Date: Mar. 14, 2024

(54) Smart Wearable IOT Device for Health Tracking, Contact Tracing and Prediction of Health Deterioration

(71) Applicant: NEW YORK UNIVERSITY, New York, NY (US)

(72) Inventors: Seyed Farokh Atashzar, New York, NY (US); Yao Wang, Livingston, NJ (US); Daniel Sterman, Radnor, PA (US)

(21) Appl. No.: 18/508,879

(22) Filed: Nov. 14, 2023

Related U.S. Application Data
(63) Continuation-in-part of application No. PCT/US22/29440, filed on May 16, 2022.
(60) Provisional application No. 63/188,587, filed on May 14, 2021.

(51) Int. Cl. A61B 5/0205; A61B 5/00

(52) U.S. Cl. CPC A61B 5/0205; A61B 5/0015; A61B 5/6814; A61B 5/6822; A61B 5/6831; A61B 5/6832; A61B 2560/0247; A61B 2560/0406; A61B 2560/045; A61B 2560/0462; A61B 2560/0475; A61B 2560/06; A61B 2562/0204; A61B 2562/0219; A61B 2562/0223; A61B 2562/0257; A61B 2562/0271

(57) ABSTRACT

The present invention provides a smart wearable IOT device configured to be worn by a subject on the body and is capable of continuously tracking and reporting a plurality of vital signs and physical activities of the user, including but not limited to sound and vibrational dynamics due to breathing and coughing (from which we can derive the breathing and coughing types and features), body temperature, peripheral oxygen saturation (SpO2), heart rate, and fatigue-related mobility, physical ability, step count, photoplethysmogram (PPG), ECG, heart rate, heart rate variability, etc. In one aspect, the wearable system will be able to measure the distance with different users through the transmit and receive of Bluetooth low energy signals or WiFi to trace contacts between different wearers. The system will be able to record the data locally on a SD card and also will be able to upload on a server.

### CROSS-REFERENCE TO RELATED APPLICATIONS

This application is a continuation-in-part of International Application No. PCT/US2022/029440, filed May 16, 2022, which claims priority to U.S. Provisional Patent Application No. 63/188,587 filed May 14, 2021, each of which is incorporated herein by reference in its entirety.

### STATEMENT REGARDING FEDERALLY SPONSORED RESEARCH OR DEVELOPMENT

This invention was made with government support under Grant No. 2031594 awarded by the National Science Foundation. The government has certain rights in the invention.

### BACKGROUND OF THE INVENTION

Continual patient monitoring of certain types of patient condition becomes a necessity to assure timely intervention by a healthcare practitioner or a physician to initiate the right medical procedure or administer the required medication in a timely manner. For example, patients suffering from infectious diseases including but not limited to severe acute respiratory syndrome coronavirus 2 (SARS-COV-2), are in need of regular or frequent testing or monitoring to track evolution of symptoms and predicting (for prevention and treatment planning) adverse events. This can be done in home as part of telemedicine (if the required technology is available), or in hospitals. On the other hand, high-risk people in a clustered environment may be continuously monitored to enable early detection of infection and reduce the chance of spreading the virus to others. Further, areas of cardiology, obstetrics, neurology, psychology are some other examples where monitoring of infectious diseases, conditions related to ambulatory patients, respiratory conditions (such as sleep apnea, or asthma) enables doctors to detect or diagnose problems, that may produce only transient symptoms and, therefore, may not be evident when the patients visit the doctors' offices.

In recent years, efforts to reduce healthcare costs, alleviate staffing constraints within healthcare facilities, and provide better care for patients have led to an increase in the use of remote patient monitoring devices. The problem with these devices is that these systems often monitor a single modality, and they cannot predict with a tunable horizon of prediction the initiation of a condition. Also, existing medical grade systems are often inconvenient restricting the use for long-term uses.

The medical grade sensing systems are typically attached to the outside of a host unit/home, are not able to track the multiple symptoms of the users while following the interpersonal interaction. Medical sensors are typically wired to the circuit boards that read or drive them. This limits where it can be positioned in relationship to the host. As well, existing systems are generally stationary, and are not battery powered, requiring them to be plugged into the wall to provide a power source. Further, because of their complexity and proprietary interfaces, many are very expensive, which reduces the cost-savings benefit of these devices.

Thus, there is a pressing need in the art for the development of smart and scalable technologies that can be produced rapidly for (a) continual in-home/in-hospital quantification, objective tracking, and reporting of the symptoms, (b) detecting early health anomalies in multidimensional symptom space (c) predicting (in time) potential adverse events for every at-risk and affected individual based on data-driven computational models, and (d) following the interaction between people in a high-risk clustered population, to enable contact tracing when the underlying condition is highly infectious. The present invention meets this need.

### SUMMARY OF THE INVENTION

In one aspect, the present invention provides a smart wearable IOT neckpiece device comprising: a sensing module comprising a front side, a rear side, a top side, a bottom side, a central part, a first end and a second end, wherein the front side comprises at least one opening and wherein the sensing module comprises a charging probe; a shaft having a proximal end, a distal end and a lumen therebetween, wherein the distal end is positioned in the central part and the proximal end is positioned within the at least one opening; at least one component comprising a first opening and a second opening, wherein the second opening is in fluid communication with the proximal end and wherein the first opening is configured to have a larger diameter than the second opening; at least one microphone; a transmit circuit; and a power source. In one embodiment, the sensing module has an asymmetric front side and rear side, and wherein the front side is concave inwards with respect to a horizontal plane, and the rear side is convex outward with respect to the horizontal plane. In one embodiment, the front side is configured to follow along the curvature of a subject's neck. In one embodiment, the first opening is configured to contact a subject's skin. In one embodiment, the at least one microphone is positioned in distal end of the shaft. In one embodiment, the at least one microphone is positioned in distal end of the shaft and at least one microphone is positioned within the at least one opening for stereo configuration to configure a differential recording for ambient noise rejection. In one embodiment, the transmit circuit is positioned in the central part and is configured to transmit digital waveforms received from the at least one microphone to one selected from the group consisting of a remote computer, a smart phone, a handheld personal digital assistant (PDA) device and combinations thereof. In one embodiment, the transmit circuit comprises one selected from the group consisting of a WiFi module or a Bluetooth module or combinations thereof. In one embodiment, the power source is a rechargeable battery. In one embodiment, the charging probes are positioned on the bottom side and have magnetic interfaces. In one embodiment, the device further comprises a charging station, wherein the charging station comprises a housing unit having a cavity configured to conform to the shape of the sensing module. In one embodiment, the cavity comprises magnets strategically oriented and aligned with the charging probes, so as to force the sensing module to self-align with the charging station. In one embodiment, the device further comprises a charging station, wherein the charging station comprises a battery that connects to the sensing module through a wired connection for charging. In one embodiment, the device further comprises a securement member. In one embodiment, the securement member is selected from the group consisting of a Velcro strap, elastic strap, headband, or a double sided connection patch. In one embodiment, the securement member comprises an adhesive to secure the device to the skin.

In one aspect, the present invention provides a smart monitoring headpiece device comprising: at least one sensor module comprising one or more sensors, a battery, a wireless transceiver, a charging circuit, a data recording module, and a control unit; and a securement member. In one embodiment, the sensor module is positioned within a housing unit. In one embodiment, the housing unit comprises a flexure mechanical auto-adaptation feature that adapts to different sizes and shapes of foreheads of users.

In one embodiment, the one or more sensor is selected from the group consisting of a temperature sensor, a presence sensor, a global positioning sensor, accelerometer sensor, IMU, one or two acoustic sensor, a gyroscope sensor, a magnetic sensor, a distance sensor, a skin contact pressure sensor, SpO2 sensor, heartrate sensor, optical Plethysmography (PPG) sensor, an ECG sensor, a respiratory rate sensor, a microphone, cough detection sensor, mobility sensor, step count sensor, Bluetooth module, Bluetooth low energy sensor (and/or WiFi) for detecting social distancing between different wearer and for data transmission, and combinations thereof. In one embodiment, the one or more sensors, battery, data recording module and charging circuit, are housed outside the housing unit in an external unit. In one embodiment, the housing unit comprises a main body and two side wings, and wherein the two side wings are configured to flex and allow the headpiece device directly contact or remain proximal to skin portion of a subject's head. In one embodiment, the main body holds one or more sensors that are to be separated by a distance from the skin of the user, and wherein one or more of the side wings comprise one or more sensors that are to be in contact with the skin of the user.

In one embodiment, the device further comprises a data recording module configured to save data on a removable memory card. In one embodiment, the battery is configured to charge the sensor module and one or more sensors in the housing unit.

In one aspect, the present invention provides a system comprising: a) a neckpiece device comprising: a sensing module comprising a front side, a rear side, a top side, a bottom side, a central part, a first end and a second end, wherein the front side comprises at least one opening and wherein the sensing module comprises a charging probe; a shaft having a proximal end, a distal end and a lumen therebetween, wherein the distal end is positioned in the central part and the proximal end is positioned within the at least one opening; at least one component comprising a first opening and a second opening, wherein the second opening is in fluid communication with the proximal end and wherein the first opening is configured to have a larger diameter than the second opening; at least one microphone; a transmit circuit; a power source; and b) a headpiece device comprising: a sensor module comprising one or more sensors, a battery, a wireless transceiver, a charging circuit, a data recording module, and a control unit; and a securement member.

In one aspect, the present invention provides a smart wearable IOT neckpiece device comprising: a sensing module comprising a front side, a rear side, a top side, a bottom side, a central part, a first end and a second end, wherein the front side comprises at least one opening and wherein the sensing module comprises a charging probe; a shaft having a proximal end, a distal end and a lumen therebetween, wherein the distal end is positioned in the central part and the proximal end is positioned within the at least one opening; at least one inward or outward cone comprising a first opening and a second opening, wherein the second opening is in fluid communication with the proximal end and wherein the first opening is configured to have a larger diameter than the second opening; at least one microphone; a transmit circuit; and a power source.

In one aspect, the present invention provides a system comprising: a) a neckpiece device comprising: a sensing module comprising a front side, a rear side, a top side, a bottom side, a central part, a first end and a second end, wherein the front side comprises at least one opening and wherein the sensing module comprises a charging probe; a shaft having a proximal end, a distal end and a lumen therebetween, wherein the distal end is positioned in the central part and the proximal end is positioned within the at least one opening; at least one inward or outward cone comprising a first opening and a second opening, wherein the second opening is in fluid communication with the proximal end and wherein the first opening is configured to have a larger diameter than the second opening; at least one microphone; a transmit circuit; a power source; and b) a headpiece device comprising: a sensor module comprising one or more sensors, a battery, a wireless transceiver, a charging circuit, a data recording module, and a control unit; and a securement member.

### BRIEF DESCRIPTION OF THE DRAWINGS

The following detailed description of invention will be better understood when read in conjunction with the appended drawings. It should be understood, however, that the invention is not limited to the precise arrangements and instrumentalities of the embodiments shown in the drawings.

FIG. 1 depicts a prospective view of an exemplary smart wearable IOT (internet of things) neckpiece device of the present invention.

FIG. 2 depicts another perspective view of an exemplary smart wearable IOT neckpiece device of the present invention.

FIG. 3 depicts a top view of an exemplary smart wearable IOT neckpiece device of the present invention.

FIG. 4 depicts a prospective view of an exemplary smart wearable IOT (internet of things) neckpiece device of the present invention

FIG. 5 depicts another perspective view of an exemplary smart wearable IOT neckpiece device of the present invention.

FIG. 6 depicts a top view of an exemplary smart wearable IOT neckpiece device of the present invention.

FIG. 7A through FIG. 7C depicts an exemplary smart wearable IOT neckpiece device of the present invention. FIG. 7A depicts a top view of a bottom side of an exemplary smart wearable IOT neckpiece device. FIG. 7B depicts a top view of the front side of an exemplary smart wearable IOT neckpiece device. FIG. 7C depicts a top view of the top side of an exemplary smart wearable IOT neckpiece device.

FIG. 8A through FIG. 8C depicts an exemplary smart wearable IOT neckpiece device of the present invention. FIG. 8A depicts a top view of a bottom side of an exemplary smart wearable IOT neckpiece device. FIG. 8B depicts a top view of the front side of an exemplary smart wearable IOT neckpiece device. FIG. 8C depicts a top view of the top side of an exemplary smart wearable IOT neckpiece device.

FIG. 9 depicts an exemplary charging station for an exemplary smart wearable IOT neckpiece device of the present invention.

FIG. 10 depicts an exemplary cover of the charging station for the smart wearable IOT neckpiece device of the present invention.

FIG. 11 depicts an exemplary charging station for an exemplary smart wearable IOT neckpiece device of the present invention.

FIG. 12 depicts a perspective view of another exemplary smart wearable IOT neckpiece device of the present invention.

FIG. 13 depicts a bottom view of an exemplary smart wearable IOT neckpiece device of the present invention.

FIG. 14 depicts an exemplary smart monitoring headpiece device of the present invention.

FIG. 15 depicts an exploded view of the housing unit of an exemplary smart monitoring headpiece device of the present invention.

FIG. 16 depicts a side view of an exemplary smart monitoring headpiece device of the present invention.

FIG. 17 depicts an exemplary securement member having an adhesive configured to attach to the housing unit of an exemplary smart monitoring headpiece device of the present invention.

FIG. 18 depicts a user wearing an exemplary neckpiece and exemplary headpiece of the present invention.

FIG. 19 depicts a perspective view of a top surface and bottom surface of an exemplary housing unit for the smart monitoring headpiece device of the present invention.

FIG. 20 depicts an exemplary smart pulse oximetry ear sensor device of the present invention.

FIG. 21 depicts an exemplary smart monitoring neckpiece with an exemplary charging station, an exemplary headpiece, and a secondary device with an executable software platform of the present invention.

FIG. 22 is a flowchart depicting an exemplary method of using the smart wearable IOT device of the present invention.

FIG. 23 is a schematic depicting the personal use of an exemplary device and system of the present invention.

FIG. 24 is a schematic depicting the use of an exemplary system and device in a clustered population of high-risk individuals.

### DETAILED DESCRIPTION

It is to be understood that the figures and descriptions of the present invention have been simplified to illustrate elements that are relevant for a clear understanding of the present invention, while eliminating, for the purpose of clarity many other elements found in the field of smart wearables. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Smart Wearable IOT Devices

The present invention provides a smart wearable internet of things (IOT) device that is configured to continuously and longitudinally detect, measure, record, data log, analyze and compare with benchmarks, one or more of a user's biometrics while enabling tracking of the interpersonal interaction between multiple wearers.

The devices of the present invention may comprise one or more of: a neckpiece, headpiece, or earpiece, as further described below. In one embodiment, the neckpiece comprises a necklace. In one embodiment, the neckpiece comprises a neck patch. In one embodiment, the headpiece comprises a headband. In one embodiment, the headpiece comprises a head patch.

In one embodiment, the device or devices of the present invention measures one or more biometrics, including but not limited to, sound and vibrational dynamics of the user's breathing or coughing sound, body temperature, oxygen saturation levels, heart rate, fatigue related mobility, step count, physical ability, ECG and etc. In one embodiment, the device of present invention is configured to detect early health anomalies and predict the development of adverse events based on the past time-series data captured by the device. In one embodiment, the device of present invention may be used for telemonitoring and tele-tracking of symptoms for subjects with infectious diseases including but not limited to diseases and disorders associated with severe acute respiratory syndrome coronavirus 2 (SARS-COV-2) infection, such as COVID-19. In one embodiment, the device of present invention can be used for both in-home remote objective telemonitoring and for in-clinic uses. In one embodiment, the device of present invention is able to keep subjects with non-critical conditions at home to reduce the healthcare burden while carefully tracking the evolution of symptoms and predicting (for prevention and treatment planning) adverse events. In another embodiment, the device of present invention may be deployed to high-risk individuals even before the initiation of symptoms to embrace early diagnosis and management.

In one embodiment, the device of present invention is configured to detect the distance between the wearers to evaluate social distancing and enable contact tracing. In one embodiment, the device of present invention is enabled with means of wireless communication to upload the data to a remote server directly. In one embodiment, the device may send the data to an edge device like a smartphone. In one embodiment, the device of present invention is enabled to record all biometrics and social distancing history on a small memory card on the device. In one embodiment, the device of present invention may be deployed to track the interpersonal distance between different wearers by monitoring the distribution of the received blue-tooth low energy signal strength from one wearer to another.

The devices described herein can be used to track infected individuals, but also may track symptoms for non-infected, but high-risk, patients which can enable early detection of infection. An example is the COVID-19 pandemic which has highly impacted the communities globally by reprioritizing the means through which various societal sectors operate. Among these sectors, healthcare providers and medical workers have been impacted prominently due to the massive increase in the demand for medical services under severe circumstances. Hence, tools, such as the presently described devices, that can help with symptom tracking, diagnosis of infection and the compliance with social guidelines for infection spread prevention will have a positive impact on managing and controlling the outbreaks and reducing the excessive burden on healthcare system. This invention proposes the use of multimodal biosensors and intelligent algorithms embodied in a wearable IoMT framework for tackling this issue. With the use of the smart wearable IoMT certain biomarkers or biometrics can be tracked for detection of a serious health condition in exposed individuals ahead of time (prediction). Using embedded intelligent algorithms, a wide range of collected biomarkers or biometrics can be processed for detecting (a) multiple symptoms of infection and (b) the dynamical likelihood of contracting the infection source through tracking interpersonal interaction in cluster of high-risk individuals. Through a systematic use of the smart wearable IoMT device in various social sectors the spread of infection can be intelligently controlled helping communities as they enter the reopening phase. This invention can benefit individuals and their medical correspondents by introducing Symptom Tracking Systems (STS), and in general the cluster of high-risk population by introducing Systems for Preventive Measures (SPM).

In addition, the wearable devices and system of the present invention will be able to measure the distance with different users through the transmission of Bluetooth low energy or WiFi to trace contacts between different wearers. For example, the devices and system will utilize the processing of the time series of signals to detect and predict health anomalies. For measuring the breathing quality using the neckpiece device, the system is particularly designed with mechanical housing which minimizes the ambient noise. Also with the use of 2 microphones (at least), 2 accelerometer, and the use of a differential recording the ambient noise is rejected and the system focuses on in-body sound, minimizing the out-body sound. In one embodiment, the system comprises a memory card to locally record the data and can at the same time upload the data on the cloud. In one embodiment, the system comprises a small shallow machine learning module on the board and a deep machine learning module on the cloud to process multiple biometric time series to detect health state and predict health deterioration of the wearer.

### Neckpiece

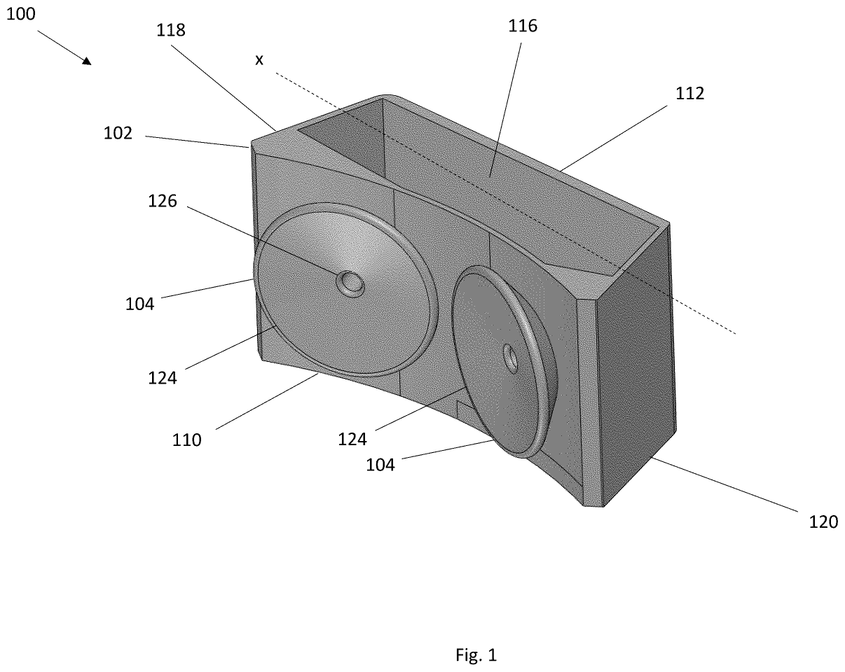

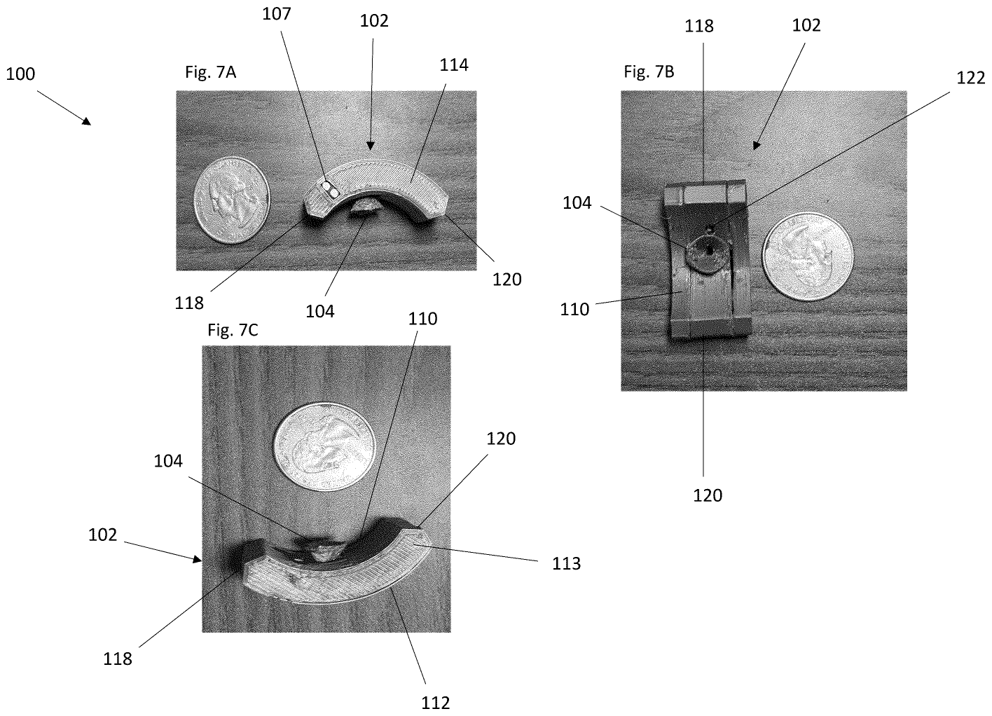

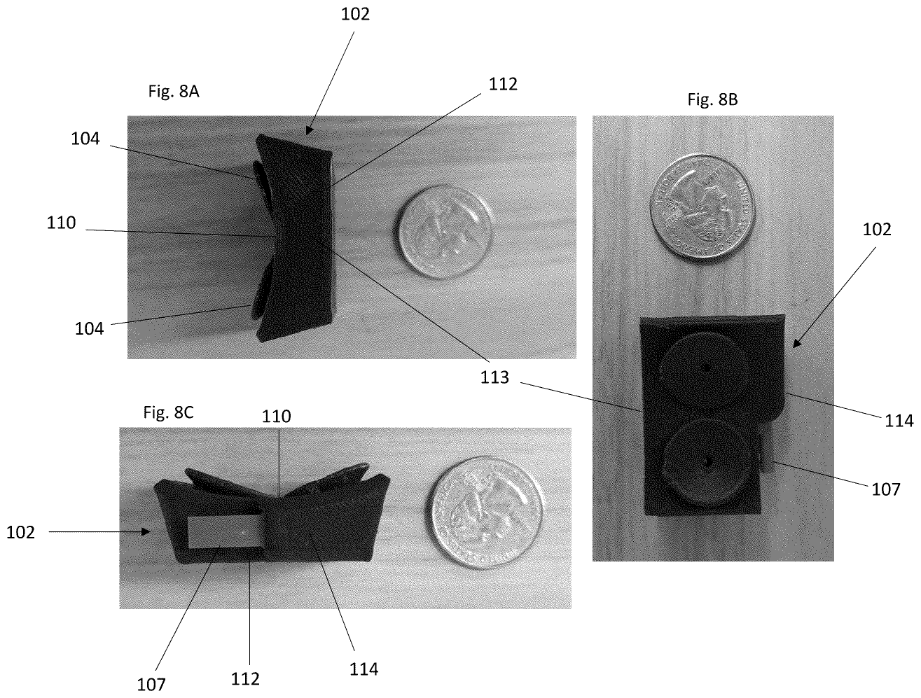

Referring now to FIG. 1 through FIG. 8, an exemplary smart wearable IOT (internet of things) neckpiece device 100 of the present invention is shown. Smart wearable IOT neckpiece device 100 comprises a sensing module 102, at least one cone 104, at least one shaft 106, a charging probe 107, a charging station 108, at least one microphone, a transmit circuit and a power source. In one embodiment, smart wearable IOT neckpiece device 100 optionally comprises a securement member. In one embodiment, smart wearable IOT neckpiece device 100 is configured to be worn around the subject's neck so that sensing module 102 rests against the suprasternal notch. In one embodiment, neckpiece device 100 is configured to be strapped around the neck of a user, similar to a necklace or a neck adorning jewelry. In one embodiment, neckpiece device 100 is configured to be adhered to the neck of a user, as a neck patch. Although referred to in some examples as at least one cone 104, it should be appreciated that at least one cone 104 may also be referred to herein as at least one component, at least one element, or at least one structure.

In one embodiment, sensing module 102 is enclosed from all sides and comprises a front side 110, a rear side 112, a top side 113, a bottom side 114, a central part 116, a first end 118 and a second end 120. In one embodiment, sensing module 102 may have asymmetric front side 110 and rear side 112. In one embodiment, front side 110 is configured to follow along the curvature of a subject's neck and is concave inwards from the horizontal plane “x”. In one embodiment, rear side 112 is convex outwards from the horizontal plane “x”. Sensing module 102 is shown having a curvature on front side 110, and the curvature on rear side 112 of sensing module 102, respectively. Curvature of front side 110 is an approximate measure of the concave nature of front side 110 of sensing module 102, while curvature of rear side 112 is an approximate measure of the convex nature of rear side 112 of sensing module 102. The approximate radii of curvature of sensing module 102 need not be equivalent, and may be varied depending upon the desired degree of curvature of the concave or convex portions of sensing module 102. In one embodiment, front side 110 is configured to follow along the curvature of a subject's neck. In one embodiment, rear side 112 may be parallel to the horizontal plane “x”.

In one embodiment, sensing module 102 comprise at least one opening 122. In one embodiment, the at least one opening 122 is positioned on front side 110. In one embodiment, the at least one opening 122 has a diameter ranging approximately between about 3-5 mm.

At least one cone 104 comprises a first opening 124 and a second opening 126. In one embodiment, first opening 124 has a diameter ranging approximately between about 1-2 cm. In one embodiment, second opening 126 has a diameter ranging approximately between about 3-5 mm. First opening 124 is configured to have a larger diameter than second opening 126. In one embodiment, first opening 124 is configured to contact the subject's skin. In one embodiment, first opening 124 is configured to be placed on the subject's suprasternal notch. At least one cone 104 is configured to direct breathing/coughing sound of the subject inward and enables the sounds to be obtained in greater amplitude. It should be appreciated that at least one cone 104 (e.g., at least one component, element, or structure) may comprise shapes or structures that direct, amplify, or otherwise impact the sound directed into sensing module 102. For example, cone 104 may be at least partially formed in one or more shapes, wherein the shapes are selected from: round, hemisphere, oval, triangle, square, rectangle, trapezoid, parallelogram, pentagon, hexagon, or the like, or cube, cylinder, cone, inward cone, outward cone, hemisphere, rectangular prism, hexagonal prism, octahedron, triangular prism, sphere, or the like.

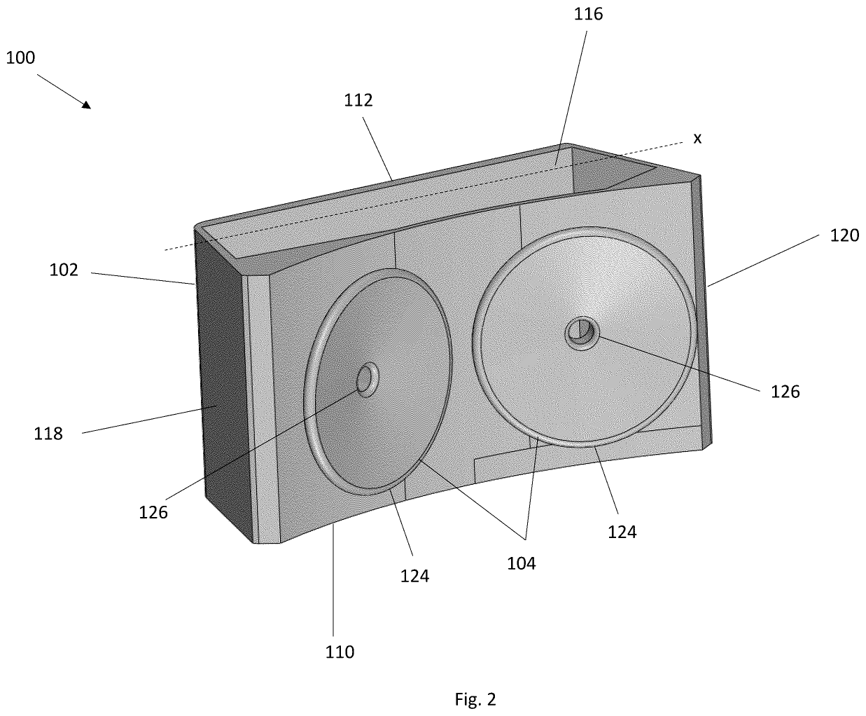

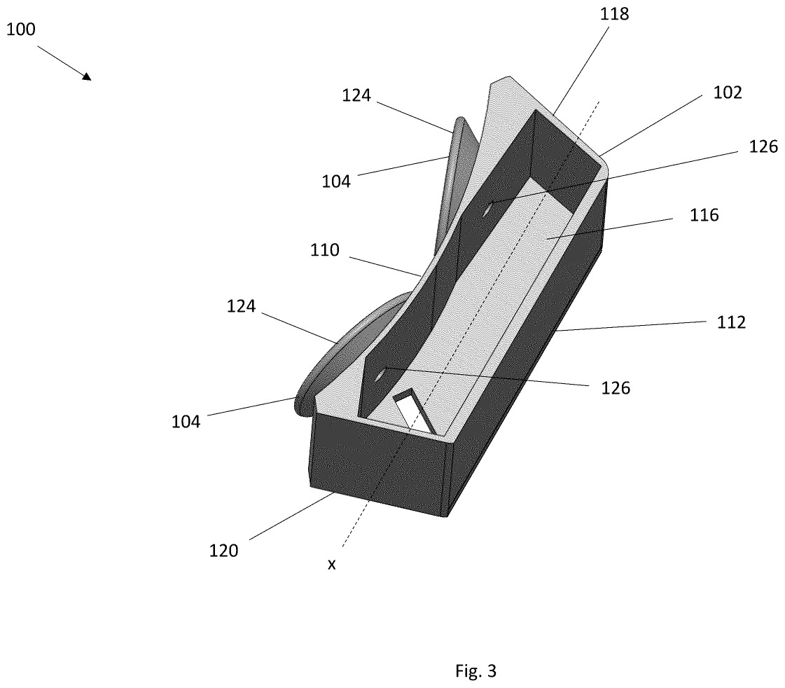

In one embodiment, neckpiece device 100 may comprise plurality of cones 104. In one embodiment, plurality of cones 104 may be positioned on front side 110. In one embodiment, neckpiece device 100 may comprise two cones 104 as depicted in FIG. 1, FIG. 2, and FIG. 3. In one embodiment, two cones 104 are positioned next to each other. In one embodiment, the distance between two cones 104 may be ranging approximately between 2 to 10 cm. In one embodiment, two cones 104 are similar in size.

In one embodiment, two cones 104 may be different in size.

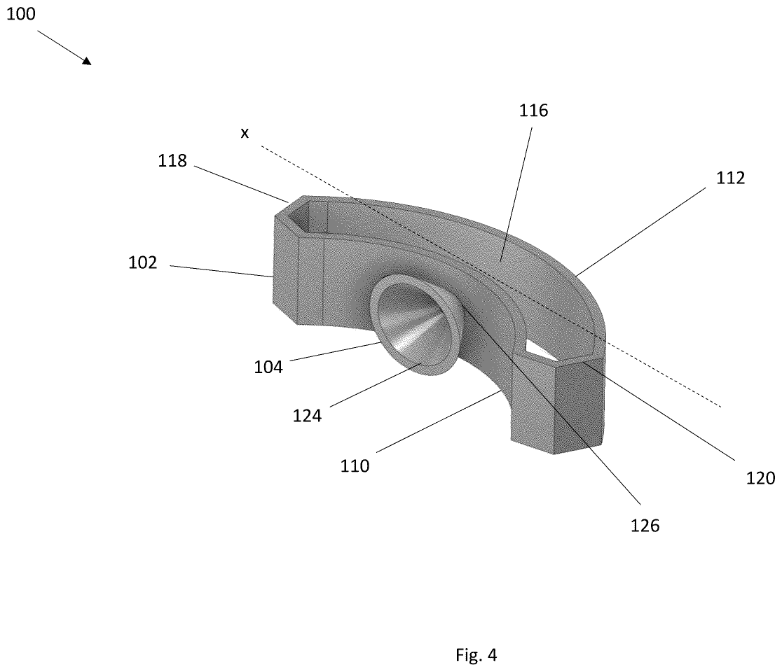

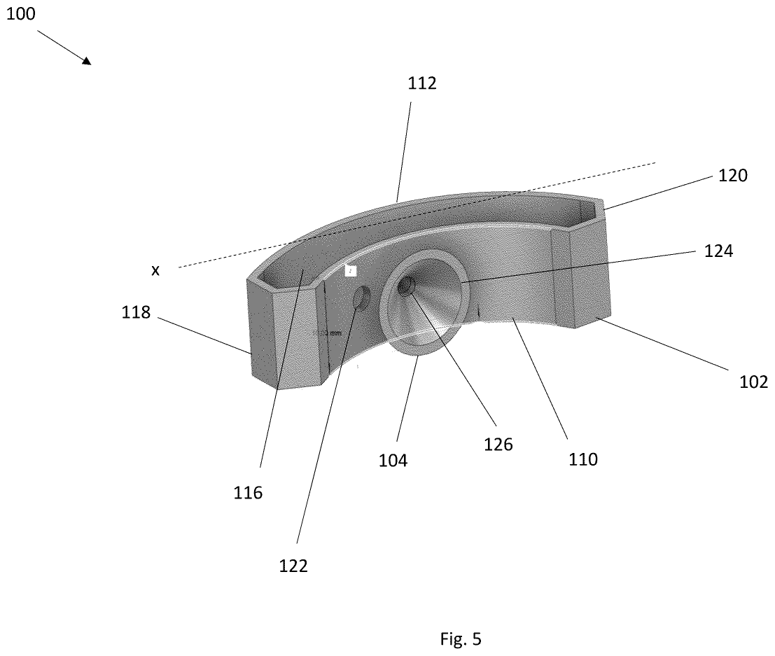

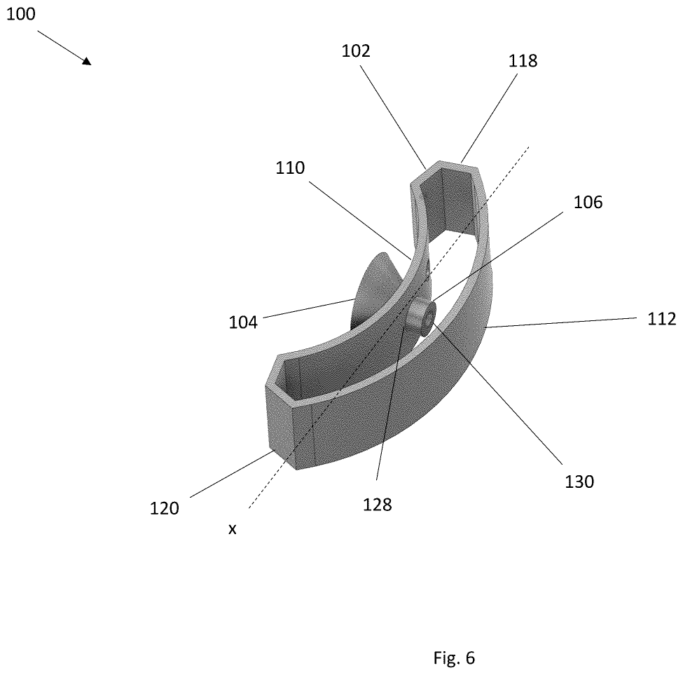

In one embodiment, neckpiece device 100 may comprise one cone 104 as depicted in FIG. 4, FIG. 5, and FIG. 6.

At least one shaft 106 comprises a proximal end 128, a distal end 130 and a lumen therebetween. In one embodiment, at least one shaft 106 has a diameter slightly smaller than second opening 126 and is configured to pass through second opening 126. In one embodiment, at least one shaft 106 is able to securely fit within second opening 126. In one embodiment, at least one shaft 106 may be fixed in place by any means known to one skilled in the art including but not limited to adhesives, snap fit and etc.

In one embodiment, sensing module 102, at least one cone 104 and at least one shaft 106 may be made of any material known to one skilled in the art including but not limited to plastics, metals, ABS, TPE, PLA, Nylon, etc. In one embodiment, sensing module 102, at least one cone 104 and at least one shaft 106 are made up of a very durable and strong plastic—Polyethylene Terephthalate (PET)—Glycol (PETG).

Proximal end 128 is fluidly connected to second opening 126. In one embodiment, shaft 106 has the same diameter as second opening 126. In one embodiment, shaft 106 and second opening 126 may be connected together by any means known to one skilled in the art. In one embodiment, shaft 106 has a diameter ranging slightly smaller than second opening 126 and is configured to securely fit inside second opening 126. In one embodiment, second opening 126 has a diameter ranging slightly smaller than at least one shaft 106 and is configured to securely fit inside at least one shaft 106.

At least one microphone is positioned at distal end 130 and is configured to pick up physiological vibration signals of the subject's breathing/coughing and transmit those sounds to a control unit. At least one cone 104 and at least one shaft 106 are configured to serve as noise reducing devices to cut out ambient noise and direct even the most minute vibrations naturally towards the at least one microphone. In one embodiment, the at least one microphone is a MEMS device. In one embodiment, the at least one microphone may be piezoelectric based. In one embodiment, the at least one microphone may be a conventional analog microphone. In one embodiment, the at least one microphone may be positioned within at least one opening 122 for stereo configuration. In one embodiment, where neckpiece device 100 comprises two cones 104, two microphones may be functionally similar to each other. In one embodiment, two microphones may be configured to function differently such that one microphone measures the in-body sound and one measures the ambient or background noise to be canceled from the first microphone.

It should be understood that shaft 106, comprising proximal end 128 and distal end 130, and the at least one opening 122, as depicted in the single-cone embodiment in FIG. 4, FIG. 5 and FIG. 6 are equally applicable to the multi-cone embodiment shown in FIG. 1, FIG. 2 and FIG. 3.

In one embodiment, neckpiece 100 measures the sound and vibration of breathing while minimizing the ambient noise through a novel differential measurement. For example, in one embodiment, one opening may be used for a second microphone to implement a novel “differential recording” of breathing sound, focusing on the in-body sound and rejecting the ambient noise related to out-body sound.

In one embodiment, the device further comprises one or more (e.g., two) accelerometer configured to touch the skin of the wearer to measure mechanical vibration associated with cough, and breathing sound in differential recording manner.

In certain embodiments of the present invention, detection of such breathing sounds resets a countdown timer such that if a sound is not detected within a given period of time, an alarm is triggered. The alarm may be either visual, auditory, sensory, or any combination thereof.

The control unit is positioned in central part 116 and is connected to a transmit circuit positioned in central part 116 that can transmit digital waveforms received from the at least one microphone to one including but not limited to a remote computer, a smart phone, a handheld personal digital assistant (PDA) device, etc. In one embodiment, the transmit circuit comprises a WiFi module or a Bluetooth module or a Bluetooth Low Energy module. Further, the persons skilled in the art can still use other wireless modules. In one embodiment, the sound signals may be sent directly to the receive circuit. In one embodiment, the wireless receive circuit of this embodiment is a Bluetooth receive module, and the receive circuit is installed in an electronic product (such as a wireless earphone, a PDA, or a computer, etc.) so that doctors and medical users can receive the data by connecting to the electronic product with the wireless receive circuit. In one embodiment, the data can be saved for future follow-ups and observations. For example, in one embodiment, the data can be saved on internal memory or on a memory card on the device. In one embodiment, Bluetooth and Wi-Fi modules on the device can also record RSSI signals strength from other wearers to calculate the distance and detect the interpersonal interaction for contact tracing.

In one embodiment communication with the edge device and cloud for uploading the data is through Wi-Fi communication. In one embodiment, communication with the edge device and cloud for uploading the data is through Bluetooth. In one embodiment the distance between multiple wearers and the distance between a wearer and some external beacons are measured through Bluetooth low energy (BLE). In one embodiment the distance between multiple wearers and the distance between a wearer and some external beacons are measured through Wi-Fi. In one embodiment the distance between multiple wearers and the distance between a wearer and some external beacons are measured through RFID.

In one embodiment, the device can communicate through BLE or Wi-Fi with other wearers (through peer-to-peer communication) or external beacons on various indoor locations to detect the location of the wearer and detect social distancing compliance. In one embodiment the system can generate a sensing cue (such as a vibration or a beeping sound), if close proximity with someone who is wearing the device and have abnormal symptoms is detected. In one embodiment, the device may record (on internal memory or memory card) and upload the history of social distancing and proximity with other wearers and beacons.

The control unit and the at least one microphone are connected to a power source positioned in central part 116. In one embodiment, power source may be a rechargeable battery. For example, the battery can be recharged when sensing module 102 is connected to charging station 108. In one embodiment, power source is a non-rechargeable battery that needs to be replaced once used. In one embodiment, sensing module 102 comprises charging probes 107 configured for charging the battery. In one embodiment, charging probes 107 may be placed on top side 113. In one embodiment, charging probes 107 may be placed on bottom side 114. In one embodiment, charging probes 107 may have magnetic interfaces. In one embodiment the electronics are placed on a flexible PCB to have better adaptability with natural curvature on the body. In one embodiment, the device will have a small SD card for recording the signals.

In one embodiment, the securement member extends from first end 118 of sensing module 102 and attaches to second end 120. In one embodiment, the securement member is long enough to pass behind the subject's neck and to hold sensing module 102 in proper position. This ensures that the sensing module 102 is placed in approximately the same position for each measurement made on a particular subject, and that it is held in proper position to acquire the relevant bioelectric signals, as explained more fully below. In one embodiment, the securement member could be split in the middle, with flexible yet shape-retaining “branches” extending from first end 118 and second end 120 of the sensing module 102 so as to pass behind the subject's neck, but not connect, much like a physician's stethoscope. The securement member can attach to sensing module 102 by any means readily known in the art, including but not limited to adhesives, clamps, snap fits, etc. In one embodiment, securement member comprises a Velcro (hook and loop) strap. In one embodiment, securement member comprises an elastic strap. In one embodiment, securement member comprises a double sided connection patch. In one embodiment, the securement member comprises an adhesive surface. For example, in one embodiment, the securement member comprises a double-sided sticky surface which (a) plays the role of a drum for the air chamber (the cone) and (b) secure the system on the skin. In one embodiment, the device further comprises a securement member that holds the neckpiece at its appropriate location. In one embodiment, the securement member comprises a headband. In one embodiment, the headband comprises a pocket, such that the sensing module 102 would fit within the pocket. In one embodiment, the sensing module 102 is secured to the headband using an adhesive.

In one embodiment, sensing module 102 may further comprise one or more sensors. Each sensor may be configured to capture, detect, and/or gather raw data characterizing a physiological or environmental parameter. Sensors may include, but are not limited to at least one of: a temperature sensor, an accelerometer sensor, a biometric sensor, a heart rate sensor, a blood glucose sensor, an oximeter sensor, an inertial measurement unit (IMU) and the like. Combining readings from one or more sensors enables smart monitoring neckpiece device to provide a number of useful data, including but not limited to a user's gait speed, head rotation angles, upper body inclination angles, stability of movement, orientation, location, temperature, pulse, blood oxygen level, breathing rate, blood pressure, coughing pattern, coughing severity, coughing rate, cough type, and the like and the like.

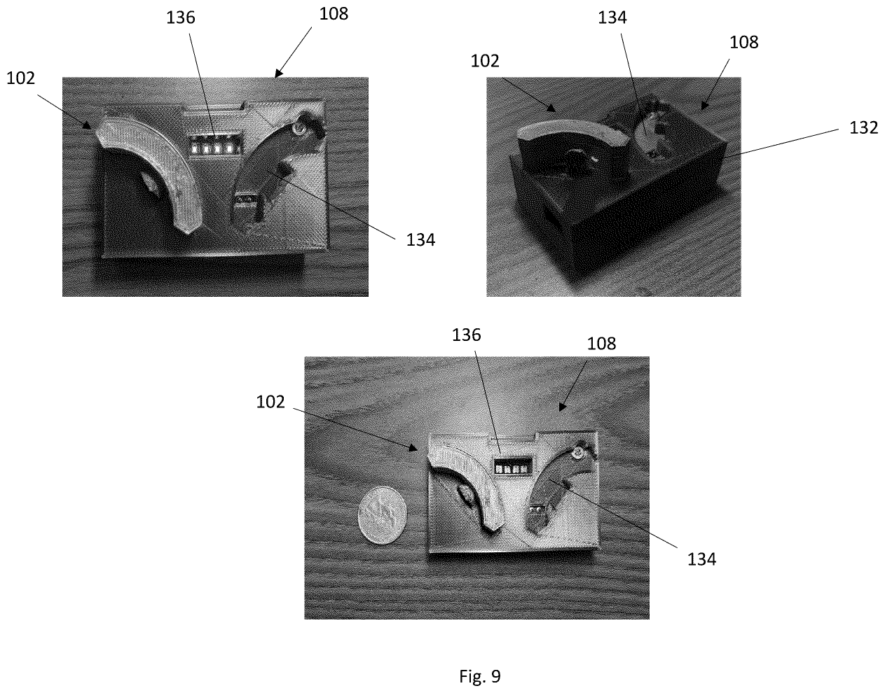

Referring now to FIG. 9, an exemplary charging station 108 is shown. In one embodiment, charging station 108 comprises a housing unit 132 having a battery and additional charging circuitry positioned within configured to quickly and easily charge sensing module 102. In some embodiment, charging station 108 may use a wired connection including but not limited to a universal USB port to charge an internal rechargeable battery and/or transfer data to an external device such as a laptop or mobile phone. In one embodiment, the battery positioned within the housing unit may be able to hold up 60 hours of battery for use. In one embodiment, charging station 108 may comprise a cavity 134 which is configured for receiving sensing module 102. Cavity 134 conforms to the shape of sensing module 102 and is configured to secure sensing module 102 in place. In one embodiment, cavity 134 comprises magnets strategically oriented and aligned with charging probes 107, so as to force sensing module 102 to self-align with charging station 108. Attachment between charging probes 107 and magnets initiates battery charging. In one embodiment, the attachment between charging probes 107 and magnets provide a force that holds sensing module 102 in charging station 108. In another embodiment, sensing module 102 may contain one or more electromagnets positioned on top side 113 or bottom side 114. Charging station 108 for charging and data transmission may also contain an electromagnet and/or a permanent magnet. In one embodiment, sensing module 102 could only turn on its electromagnet when it is close to charging station 108. In one embodiment, sensing module 102 may detect proximity to charging station 108 by looking for the magnetic field signature of a permanent magnet in charging station 108 using a magnetometer. In one embodiment, the electromagnet could be reversed, creating a force that repels sensing module 102 from charging station 108 either when sensing module 102 doesn't need to be charged, synced, or when it has completed syncing or charging. In some embodiments, charging station 108 may include the electromagnet and may be configured (e.g., a processor in the charger or dock may be configured via program instructions) to turn the electromagnet on when sensing module 102 is connected for charging (the electromagnet may normally be left on such that sensing module 102 that is placed on charging station 108 is drawn against charging station 108 by the electromagnet, or the electromagnet may be left off until charging station 108 determines that sensing module 102 has been placed on charging station 108, e.g., through completion of a charging circuit, etc., and then turned on to draw sensing module 102 against the charger. Upon completion of charging (or of data transfer, if the charger is actually a data transfer cradle or a combined charger/data transfer cradle), the electromagnet may be turned off (either temporarily or until sensing module 102 is again detected as being placed on charging station 108) and sensing module 102 may stop being drawn against charging station 108. In such embodiments, it may be desirable to orient the interface between sensing module 102 and charging station 108 such that, in the absence of a magnetic force generated by the electromagnet, sensing module 102 would fall off of charging station 108 or otherwise shift into a visibly different position from the charging position (to visually indicate to a user that charging, or data transfer is complete).

In one embodiment, housing unit 132 may further comprise a plurality of LEDs 136 configured to show the charging status of sensing module 102. In one embodiment, plurality of LEDs 136 may turn on as the charging of sensing module increases. In one embodiment, the plurality of LEDs 136 may illuminate different colors to indicate charging status. For example, a green LED may be turned on to indicate sensing module is fully charged, a red LED may be turned on to indicate sensing module 102 needs charging, etc. In one embodiment, a blinking light may indicate that sensing module 102 is not correctly positioned on charging station 108. It will be understood by those of ordinary skilled in the art that any other LED configuration or alarming means may be used to notify the subject about the charging status and positioning of sensing module 102 on charging station 108.

In some embodiment, charging station 108 may use a wired connection including but not limited to a universal USB port to charge an internal rechargeable battery and/or transfer data to an external device such as a laptop or mobile phone.

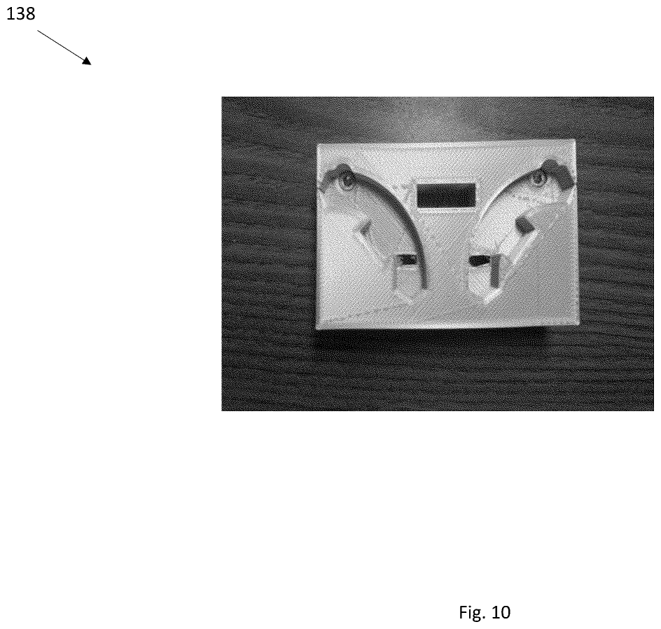

Referring now to FIG. 10, charging station 108 may optionally comprise a cover 138 configured to make sure there is precise contact between charging probe 107 on sensing module 102 and magnets on charging station 108. In one embodiment, charging station 108 and cover 138 may be made from a material including but not limited to a plastic. In one embodiment, charging station 108 and cover 138 may be made from PLA. In one embodiment, charging station 108 and cover 138 may be made from polyethylene Terephthalate glycol (PETG).

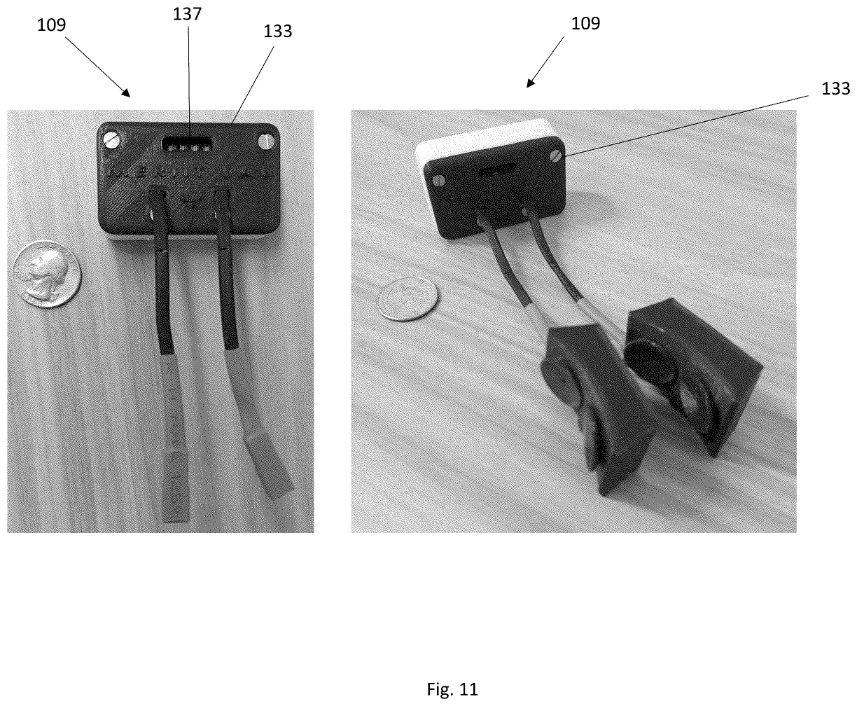

Referring now to FIG. 11, another exemplary charging station 109 is shown. In one embodiment, charging station 109 comprises a housing unit 133 having a battery and additional charging circuitry positioned within configured to quickly and easily charge sensing module 102. In one embodiment, the battery may connect to sensing module 102 by any means known to one skilled in the art to allow recharging sensing module 102. In one embodiment, the battery may connect to sensing module 102 through a wired connection.

In one embodiment, housing unit 133 may further comprise a plurality of LEDs 137 configured to show the charging status of sensing module 102. In one embodiment, plurality of LEDs 137 may turn on as the charging of sensing module increases. In one embodiment, the plurality of LEDs 137 may illuminate different colors to indicate charging status. For example, a green LED may be turned on to indicate sensing module is fully charged, a red LED may be turned on to indicate sensing module 102 needs charging, etc. In one embodiment, a blinking light may indicate that sensing module 102 is not correctly positioned on charging station 108. It will be understood by those of ordinary skilled in the art that any other LED configuration or alarming means may be used to notify the subject about the charging status and positioning of sensing module 102 on charging station 109.

In one embodiment, charging station 109 may allow charging of at least one sensing module 102 at a time. In one embodiment, charging station 109 may be connected and charging two sensing module 102 at one time.

In some embodiment, charging station 109 may use a wired connection including but not limited to a universal USB port to charge an internal rechargeable battery and/or transfer data to an external device such as a laptop or mobile phone.

The various components of the present invention described above can be constructed using any suitable method known in the art. The method of making may vary depending on the materials used. For example, components substantially comprising a metal may be milled from a larger block of metal or may be cast from molten metal. Likewise, components substantially comprising a plastic or polymer may be milled from a larger block, cast, or injection molded. In some embodiments, the devices may be made using 3D printing or other additive manufacturing techniques commonly used in the art.

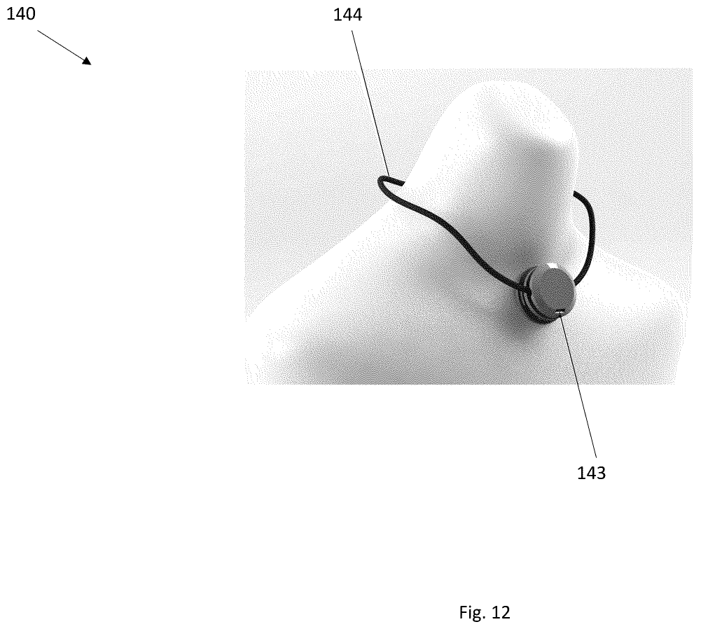

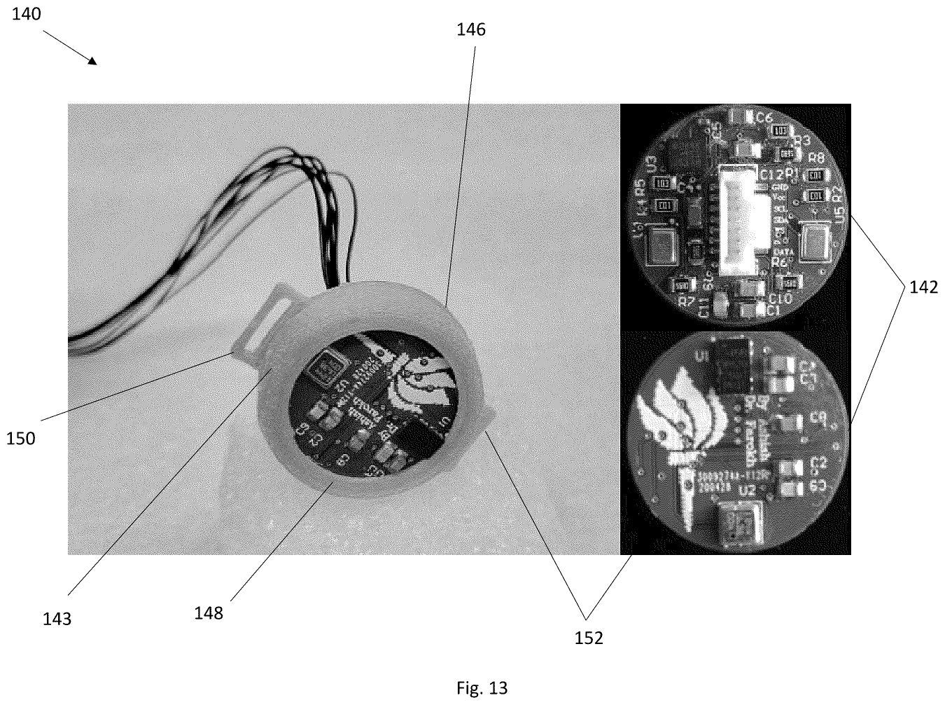

Referring now to FIG. 12 and FIG. 13, another exemplary smart wearable internet of things (IOT) neckpiece device 140 of the present invention is shown. Wearable IOT necklace device 140 comprises a sensing module 142, a housing unit 143 and a securement member 144. In one embodiment, housing unit 143 comprises a top side 146 and a bottom side 148. Bottom side 148 is configured to rest against the suprasternal notch, similar to a necklace or a neck adorning jewelry from. Top side 146 comprises a first end 150 and a second end 152. In one embodiment, securement member 144 extends from first end 150 of housing unit 143 and attaches to second end 152. Securement member 144 is structurally and functionally similar to securement member described above with smart wearable IOT neckpiece device 100.

In one embodiment, sensing module 142 and housing unit 143 may be designed in a circular format to avoid the edges and provide ease of use for the subject upon wearing. In one embodiment, sensing module 142 and housing unit 143 may have any other shape known to one skilled in the art including but not limited to oval, etc. In one embodiment, housing unit 143 may be made of any material known to one skilled in the art including but not limited to plastics, metals, ABS, TPE, PLA, Nylon, etc.

Sensing module 142 comprises a plurality of sensors, at least one microphone, at least one accelerometer, and a multipin connector. The plurality of sensors may be configured to capture, detect, and/or gather raw data characterizing a physiological or environmental parameter. In one embodiment, sensor module 142 comprises at least two microphones. In one embodiment, sensor module 142 comprises at least two accelerometers. In one embodiment, the at least two microphones and/or at least two accelerometers of the sensing module are used in a differential recording mode to reject ambient noise and maximize signal to noise ratio. In one embodiment, one accelerometer/microphone which is not in touch with the skin, is configured to only measure the ambient noise, whereas the other unit is configured to measure the body sound plus the ambient noise. The differential recording will result in enhancement of the signal quality.

In one embodiment, the plurality of sensors may include, but are not limited to at least one of: a temperature sensor, an accelerometer sensor, a biometric sensor, a heart rate sensor, a blood glucose sensor, an oximeter sensor, blood pressure sensor, electrocardiogram sensor, an inertial measurement unit (IMU) and the like. The at least microphone is placed on bottom side 148 and is configured to pick up physiological vibration signals of the subject's breathing/coughing and transmit those sounds to a central processing unit (CPU). In one embodiment, two microphones may be used in stereo configuration. In one embodiment, the at least one microphone is a MEMS device.

In one embodiment, the CPU is positioned on the chest of a subject and is configured to communicate with sensing module 142 via a 7 pin clasp connector. In one embodiment, the CPU is configured to monitor live data, run various algorithms, remotely control sensing module 142 and record the data.

In one embodiment, sensing module 142 and/or the CPU stores, monitors, and transmits data as previously described. For example, in one embodiment, sensing module 142 and/or the CPU comprises a Bluetooth module, Wi-Fi module, RFID module, or the like for data communication and to determine the distance between multiple wearers or between a wearer and an external beacon.

### Headpiece Device

The present invention provides a smart monitoring headpiece device that is configured to continuously and longitudinally detect, measure, record, data log, analyze and compare with benchmarks, one or more of a user's biometrics. In one embodiment, smart monitoring headpiece device is used in combination with the smart wearable internet of things (IOT) neckpiece device as described above to accurately and objectively track multiple vital symptoms of a subject. In one embodiment, the headpiece device comprises one or more sensors, control units, data communication modules, as described above with respect to the neckpiece device. In one embodiment, the headpiece device comprises a headband. In one embodiment, the headpiece device comprises a head patch.

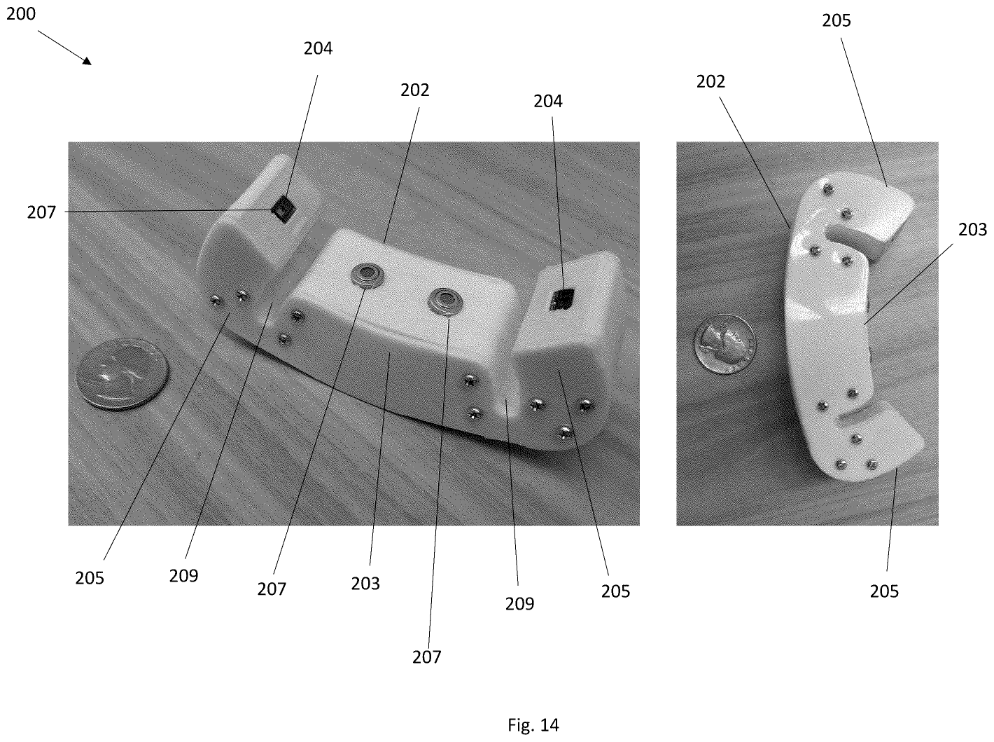

Referring now to FIG. 14 through FIG. 17, an exemplary smart monitoring headpiece device 200 of the present invention is shown. Smart monitoring headpiece device 200 comprises a housing unit 202 having at least one sensing module 204 and a securement member 206.

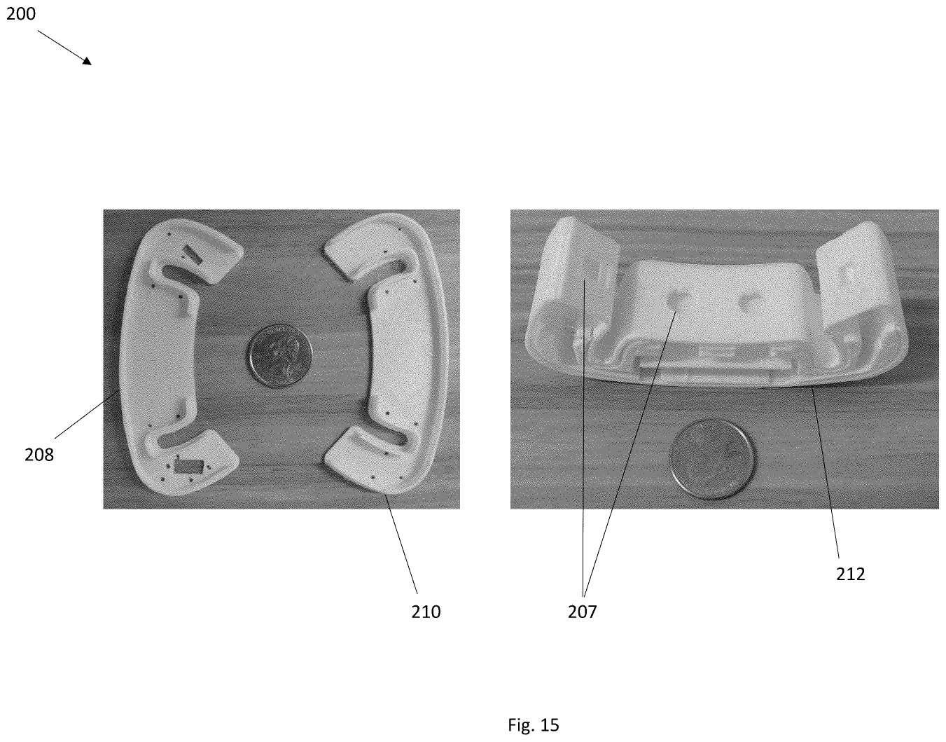

Housing unit 202 is adapted to be positioned on the subject's forehead. For example, housing unit 202 may be placed on central, middle or lateral forehead and it may be distributed symmetrically or may be only at one side. Housing unit 202 comprises a main body 203 and two side wings 205, wherein main body 203 is separated from side wings 205 by two grooves 209. Grooves 209 are configured to allow side wings 205 to flex and allow headpiece device 200 to conform to any head shape. In one embodiment, housing unit 202 may be made using 3D printing or other additive manufacturing techniques commonly used in the art. In one embodiment, housing unit 202 may have a flexure design (mechanical auto-adaptation) that allows the device to fit the forehead size and shape of different users with different forehead anatomy. In one embodiment, housing unit 202 may be made from any applicable material including but not limited to high quality PETG. In one embodiment, housing unit 202 may comprise a top body 208, a bottom body 210 and a middle body 212 (FIG. 15). In one embodiment, top body 208 and bottom body 210 can be snapped onto middle body 212.

In one embodiment, housing unit 202 may have at least one opening 207 positioned on middle body 212. In one embodiment, the at least one opening 207 may have any shape including but not limited to circular, rectangular, square, etc. In one embodiment, at least one opening 207 may be positioned anywhere on housing unit 202. In one embodiment, at least one opening 207 may be positioned on main body 203. In one embodiment, at least one opening 207 may be positioned on two side wings 205. In one embodiment, the at least one opening 207 may be configured for allow sensors including but not limited to infrared temperature sensor, SpO₂ and PPG to be directly in contact or remain proximal to the skin portion of the subject's body. In certain instances, the at least one opening 207 are configured to allow for needed distance between the skin and sensor for contactless sensors, such as contactless temperature sensors. In certain embodiments, housing unit 202 comprises at least one opening 207 on main body 203 configured to provide distance between the sensor and skin for contactless sensors, and further comprises at least one opening 207 on one or more of side wings 205 for sensors requiring direct skin contact.

In one embodiment, at least one sensor module 204 is positioned anywhere within housing unit 202. In one embodiment, at least one sensor module 204 may be positioned within housing unit 202, such that it would allow direct contact or remain proximal to the skin portion of the subject's body. In one embodiment, at least one sensor module is positioned such that it provides a distance from the skin needed for certain contactless sensors. In one embodiment, at least one sensing module 204 may be positioned on two side wings 205. In one embodiment, at least one sensing module 204 may be positioned on main body 203. In one embodiment, at least one sensor module 204 comprises one or more sensors, a battery, a wireless transceiver, a charging circuit, a data recording module, and a control unit. Each sensor may be configured to capture, detect, and/or gather raw data characterizing a physiological or environmental parameter. Sensors may include, but are not limited to at least one of: a temperature sensor, a presence sensor, a global positioning sensor, an accelerometer sensor, Inertial Measurement Unit (IMU), acoustic sensor, a gyroscope sensor, a magnetic sensor, a distance sensor, a skin contact pressure sensor, a light sensor, a heart rate sensor, a microphone, an optical photoplethysmogram (PPG) sensor, SpO2 sensor, IMU, ECG, a respiratory rate sensor, a cough detection sensor, mobility sensor, a step count sensor, a Bluetooth low energy sensor or Wi-Fi for detecting social distancing between different wearers and for data transmission, Wi-Fi and the like. In certain embodiments, headpiece 200 comprises multiple sensors of the same time. In certain instances, redundant and parallel recording (multiple sensors of one type) may be considered to enhance the signal quality and robustness allowing higher reliability of the system, for example when one of the sensors fails or does not secure a good signal quality.

In one embodiment, at least one sensor module 204 comprises a distance transceiver. In one embodiment, at least one sensor module 204 comprises various sensor slots to easily add or remove sensors based on the user's needs. In one embodiment, at least one sensor module 204 may include an Arduino Nano 33 BLE Sense, wherein the Arduino comprises a body temperature sensor from a very sensitive and accurate IR temperature sensor and 9-Axis Inertia measurements including 3 accelerometer, 3 gyroscope and 3 magnetometer readings. However, the device is not limited to the Arduino Nano 33 BLE Sense. Rather, the device may comprise any microcomputer and any applicable sensors. In one embodiment headpiece device 200 may have a data logger which is able to record the data on a small removable memory card.

In one embodiment, housing unit 202 may comprises one or more sensors. In one embodiment, one or more sensors may be positioned anywhere within housing unit 202. In one embodiment, one or more sensors may be positioned in main body 203. In one embodiment, one or more sensors may be positioned in two side wings 205. In one embodiment, one or more sensors may be positioned within housing unit 202 and facing at least one opening 207 of housing unit 202 such that it would allow one or more sensors to directly contact or remain proximal to the skin portion of the subject's body.

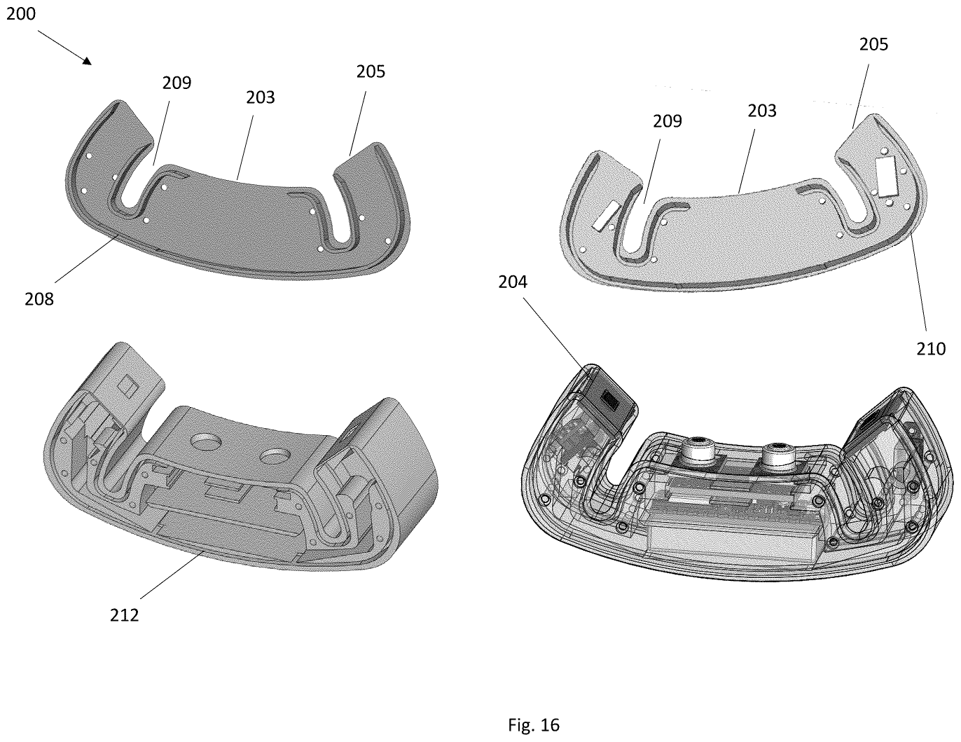

In one embodiment, the one or more sensors in housing unit 202 is an oximeter sensor configured to measure various blood flow characteristics including, but not limited to, the blood-oxygen saturation of hemoglobin in arterial blood, the volume of individual blood pulsations supplying the tissue, and the rate of blood pulsations corresponding to each heartbeat of a user. Measurement of these characteristics has been accomplished by use of a non-invasive sensor which scatters light through a portion of the user's tissue where blood perfuses the tissue, and photoelectrically senses the absorption of light in such tissue. The amount of light absorbed is then used to calculate the amount of blood constituent being measured. In one embodiment, a SparkFun pulse oximeter and heart sensor is used. In one exemplary embodiment, one or more sensors positioned on main body 203 as depicted in FIG. 16 may be a temperature sensor or any other sensor that may require some distance from the skin to secure high quality recording. In one exemplary embodiment, one or more sensors positioned on two side wings 205 as depicted in FIG. 16 may be a SpO₂ sensor and/or heartrate sensor. In one embodiment, each side wing 205 may comprise the same type of sensor. In certain instances, recording at two locations increases the signal to noise ratio and the robustness of the system, such as instances when one sensor fails during certain episodes of recording.

In one embodiment, a battery is placed inside housing unit 202. In one embodiment, the battery is configured to power the at least one sensor module 204 and one or more sensors positioned within housing unit 202. Battery can be any suitable battery, such as a rechargeable battery or a replaceable battery. Embodiments comprising a rechargeable battery can further comprise one or more features to enable recharging, such as a cable port for connecting to a power source. In certain embodiments, recharging is performed wirelessly by way of one or more inductive charging coils.

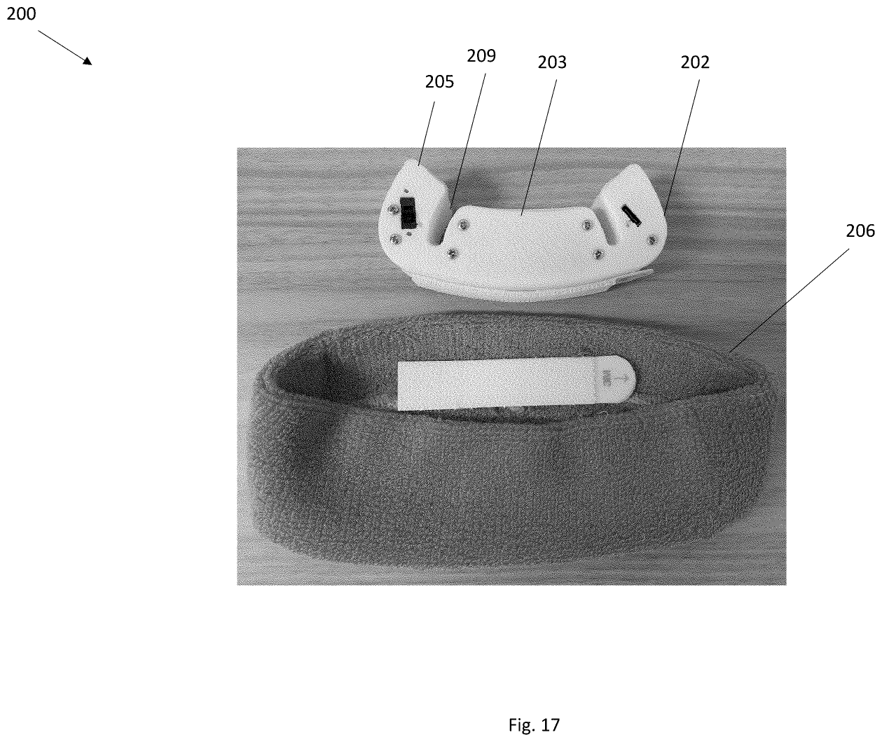

In one embodiment, securement member 206 may include an elastic segment of a specific length, to provide a specific spring force once stretched. In one embodiment, securement member 206 may be attached to housing unit by any means known to one skilled in the art. In one embodiment, securement member 206 may be attached to housing unit 202 with an adhesive (FIG. 17). In one embodiment, securement member 206 may be in a form of a headband having a pocket within, such that housing unit 202 would fit within the pocket. In one embodiment, securement member 206 may be attached to one side wing 205, wrap around the subject's head and fastened to the second side wing 205 using an adjustable closure mechanism, such as for example a hook and loop closure mechanism.

In one embodiment, securement member 206 may have an adjustable length. In one embodiment, securement member 206 may be a Velcro strap. In one embodiment, securement member 206 may be adjusted for use with any size subject by using an adjustable closure mechanism, such as for example a hook and loop closure mechanism. Alternately, in one embodiment, securement member 206 may be provided in varying sizes, depending on the general size of the subject's head; for example, using a small securement member 206 for a neonate, a larger one for a child and an even larger one for an adult wearer. In one embodiment, the different head sizes of the subject are accommodated by providing a suite of different sized headpieces; starting with the smallest and graduating to larger sized ones; all having common features as described herein.

Alternatively, in one embodiment, securement member 206 may be two piece, wherein the first piece is attached to one side wing 205 at one end and has a closure device on another end and the second piece is attached to second side wing 205 from one end and has a closure device on another end and wherein the closure devices are configured to attach together by any means known to one skilled in the art, including but not limited to a snap fit, a hook and loop, etc.

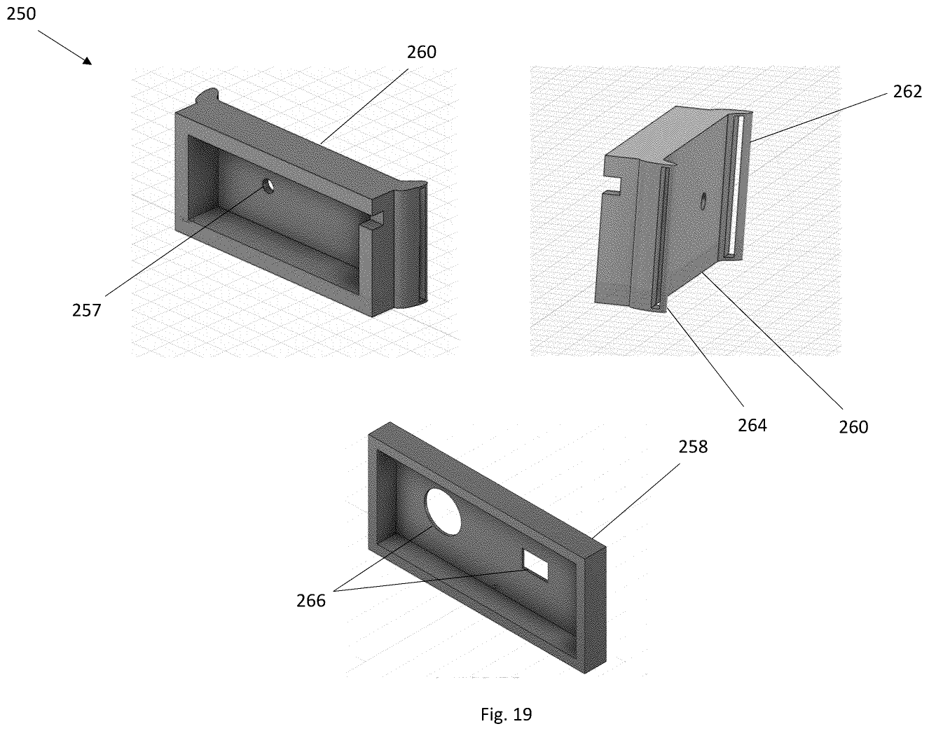

Referring now to FIG. 19, another exemplary smart monitoring headpiece device 250 of the present invention is shown. Smart monitoring headpiece device 250 comprises a housing unit 252 having at least one sensing module 254 and a securement member 256. Housing unit 252 comprises a top surface 258, a bottom surface 260 and one or more sensor positioned inside housing unit 252. Bottom surface 260 comprises a first end 262 and a second end 264. First end 262 and second 264 comprises at least one opening configured to allow securement member 256 to pass through. In one embodiment, bottom surface 260 may have at least one opening 257. In one embodiment, the at least one opening 257 may have any shape including but not limited to circular, rectangular, square, etc. In one embodiment, top surface 258 comprises at least one opening 266 configured to allow at least one sensor module 254 and one or more sensors in housing unit 252 to directly contact or remain proximal to the skin portion of the subject's body. In one embodiment, the at least one opening 266 may have any shape including but not limited to circular, rectangular, square, etc.

In one embodiment, one or more sensors may be positioned outside of housing unit 252 and inside an external unit 255. In one embodiment, external unit 255 may be secure anywhere on securement member 256.

At least one sensor module 254 and securement member 256 is structurally and functionally similar to at least one sensor module 204 and securement member 206 described elsewhere herein.

In one embodiment, smart monitoring headpiece device 200 and 250 comprise a control unit electrically connected to at least one sensor module 204 or at least one sensor module 254 and one or more sensors positioned within housing unit 202 or housing unit 252 and communication devices (for example, wireless transceiver). In one embodiment, smart monitoring headpiece device 200 and 250 may further comprise a memory. In one embodiment, the memory can include one or more non-transitory computer-readable media.

The control unit is configured to receive and interpret physiological and environmental data. The control unit may compare a representative value with a preset or adjustable value stored in the memory to further characterize a physiological condition of the body. For example, control unit may compare the body temperature of a subject with a preset or adjustable threshold value stored in the memory to determine that the temperature of the body is relatively too high or too low. In another example, control unit may detect a rapid rise in pulse when comparing a change in pulse rate versus a threshold value stored in the memory.

Combining readings from one or more temperature sensors, presence sensors (which certifies the contact between the sensor and the skin), global positioning sensors, accelerometer sensors, gyroscope sensors, magnetic sensors, distance sensors, pressure sensors, light sensors, respiratory rate sensor, PPG sensor, SpO2 sensor, ECG, acoustic sensor, microphone, and the like enables smart monitoring headpiece device 200 to provide a number of useful data, including but not limited to a user's gait speed, head rotation angles, upper body inclination angles, stability of movement, orientation, location, temperature, pulse, blood oxygen level, breathing rate, coughing pattern, coughing severity, coughing rate, cough type, step count, mobility, and the like and the like.

In one embodiment, the wireless transceiver can be any suitable transceiver for wirelessly transmitting and receiving signals, including one or more of a Bluetooth transceiver, Bluetooth low energy, WiFi transceiver, near field communication transceiver, mobile transceiver (e.g., 3G, 4G, 5G, etc.), and the like.

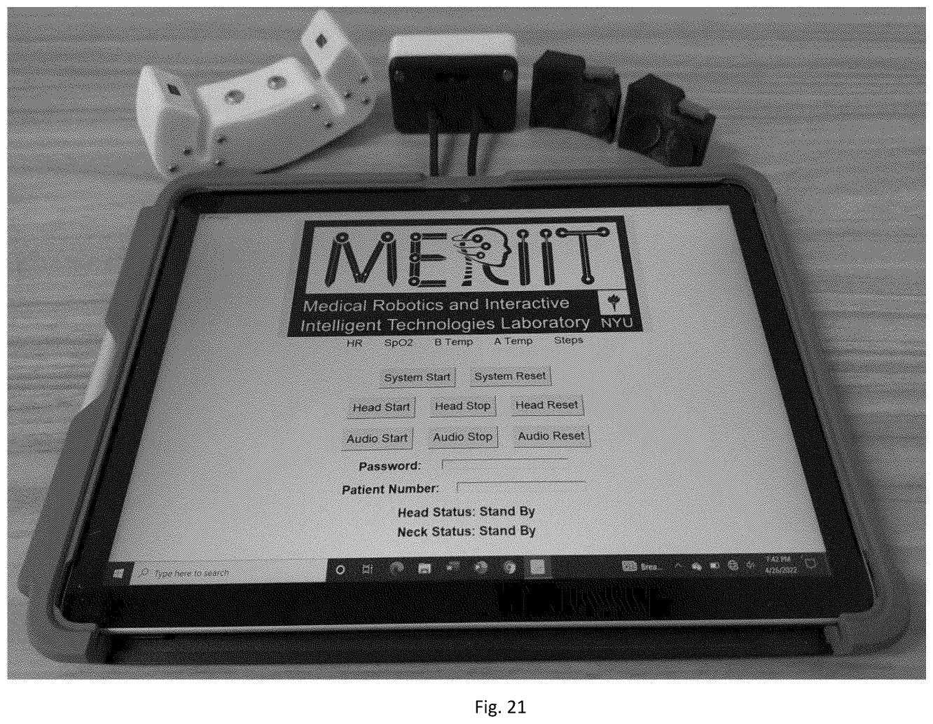

As described above, the control unit and memory are electrically connected to one or more sensors, the wireless transceiver, and the battery. In one embodiment, the control unit and memory may operate in conjunction with a local or remote executable software platform, or with a hosted internet or network program or portal (secondary device) (FIG. 21). As contemplated herein, any computing device as would be understood by those skilled in the art may be used with the control unit and memory, including desktop or mobile devices, laptops, desktops, tablets, smartphones, or other wireless digital/cellular phones, televisions or other thin client devices as would be understood by those skilled in the art. In one embodiment, the control unit may comprise one or more logic cores. In some embodiments, the control unit may comprise more than one discrete integrated circuit.

In one embodiment, the control unit, in conjunction with the several components of smart monitoring headpiece device 200 and 250, is fully configured to send, receive, and interpret device signals as described herein. For example, control unit can be configured to monitor, and record signals observed by one or more sensors to the memory. Further, in one embodiment, the control unit can be configured to record some or all of the received signals from onboard components to the memory and subsequently interpret the signals. Control unit can also be configured to record received signals from the wireless transceiver to the memory and subsequently interpret the signals. Signals can also be sent to cloud storage via the wireless transceiver. Control unit may be configured to interpret the various signals as a series of data points and subsequently transmit the data points to a digital display. Control unit may further perform automated calculations based on the various signals to output information such as velocity, acceleration, orientation, angle, location, and the like, depending on the type of signals received.

In one embodiment, smart monitoring headpiece device 200 and 250 may be configured to output information to a user, to another wearable device, to a non-wearable device, or to a network according to the particular features and function of the device. In one embodiment, the device can communicate through BLE or Wifi with other wearers (through peer-to-peer communication) or external beacons on various indoor locations to detect the location of the wearer and detect social distancing compliance. In one embodiment the system can generate a sensing cue (such as a vibration or a beeping sound), if close proximity with someone who is wearing the device and have abnormal symptoms is detected. In one embodiment, the device will record (on internal memory or memory card) and upload the history of social distancing and proximity with other wearers and beacons.

### Smart Pulse Oximetry Ear Sensor

The present invention further provides a smart pulse oximetry ear sensor device that is configured to continuously and longitudinally detect, measure, record, data log, analyze and compare with benchmarks, the peripheral oxygen saturation (SpO2) levels of the subject. In one embodiment, the smart pulse oximetry ear sensor device may be used with the smart wearable internet of things (IOT) necklace neckpiece, or the smart wearable internet of things (IOT) headpiece as described above to accurately and objectively track multiple vital symptoms of a subject.

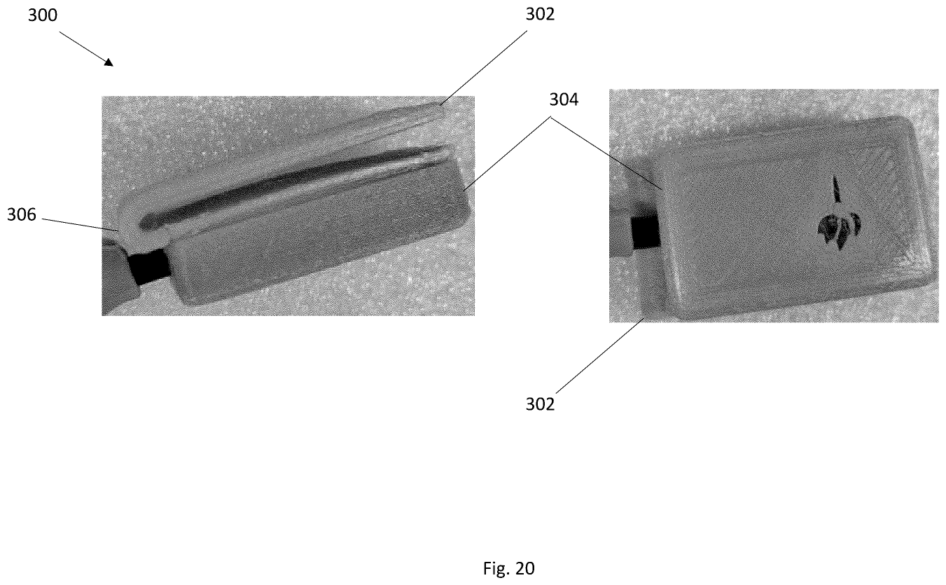

Referring now to FIG. 20, an exemplary smart pulse oximetry ear sensor device 300 of the present invention is shown. Smart pulse oximetry ear sensor device 300 comprises a first unit 302 and a second unit 304. First unit 302 and second unit 304 are two mating halves, hingedly connected at a first point 306 and are configured to be positioned on either side of the subject's earlobe. In one embodiment, one or more sensors are housed inside second unit 304. Second unit 304 comprises an inner side 308 which is configured to face one side of the user's earlobe. Second unit 304 comprises at least one opening on inner side 308 configured to directly contact or remain proximal to the skin portion of the subject's body. In one embodiment, the at least one opening may have any shape including but not limited to circular, rectangular, square, etc. In one embodiment, inner side 308 may include more than two openings.

In one embodiment, the one or more sensors in second unit 304 is an oximeter sensor configured to measure various blood flow characteristics including, but not limited to, the blood-oxygen saturation of hemoglobin in arterial blood, the volume of individual blood pulsations supplying the tissue, and the rate of blood pulsations corresponding to each heartbeat of a user. Measurement of these characteristics has been accomplished by use of a non-invasive sensor which scatters light through a portion of the user's tissue where blood perfuses the tissue, and photoelectrically senses the absorption of light in such tissue. The amount of light absorbed is then used to calculate the amount of blood constituent being measured. In one embodiment, a SparkFun pulse oximeter and heart sensor is used.

In one embodiment, a battery is placed inside second unit 304. In one embodiment, the battery is configured to power the one or more sensors positioned within second unit 304. Battery can be any suitable battery, such as a rechargeable battery or a replaceable battery. Embodiments comprising a rechargeable battery can further comprise one or more features to enable recharging, such as a cable port for connecting to a power source. In certain embodiments, recharging is performed wirelessly by way of one or more inductive charging coils.

In one embodiment, smart pulse oximetry ear sensor device 300 is connected to an external CPU via a multipin connector. In one embodiment, the CPU is configured to monitor live data, run various algorithm, remotely control sensing module 142, record the data, etc. In one embodiment, the CPU may be positioned anywhere on the subject's body. In one embodiment, the CPU may be positioned on the subject's chest.

In one embodiment, smart pulse oximetry ear sensor device 300 maybe connected to any other smart wearable device disclosed in this application. For example, smart pulse oximetry ear sensor device 300 maybe connected to smart wearable IOT neckpiece device 100 and/or headpiece device 200, wherein the device comprises a control unit positioned anywhere between the multiple devices.

### Systems

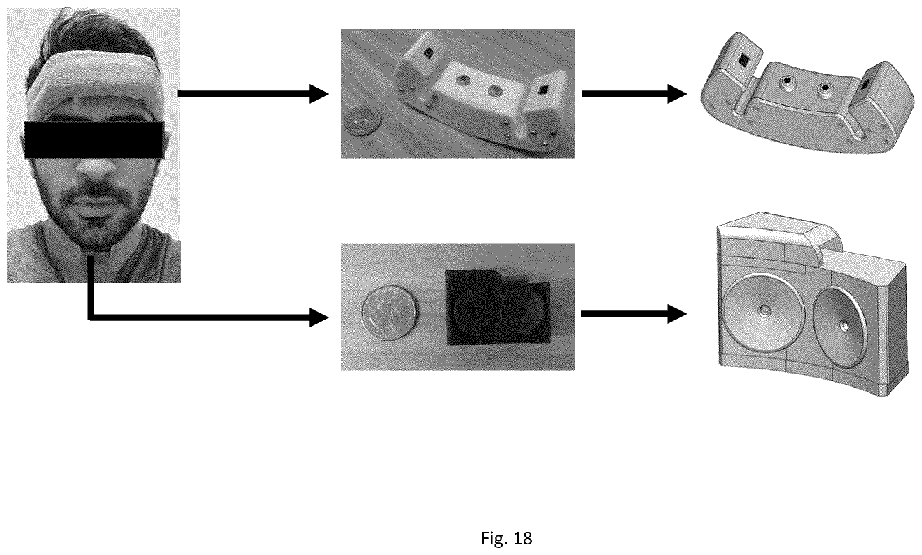

In one embodiment, the present invention comprises a system comprising a smart neckpiece device and a smart headpiece device, described above. For example, FIG. 18 depicts a user wearing both a neckpiece and a headpiece described herein.

In one embodiment, the system further comprises an ear sensor device. In one embodiment, the system comprises a control unit to communicate with each component. For example, the control unit may transmit and receive data from each component, store data, analyze data, transmit data to a remote computing device, transmit data to the cloud, and the like.

In one embodiment, the system comprises a memory card to locally record the data and can at the same time upload the data on the cloud. In one embodiment, the system comprises a small shallow machine learning module on the board and a deep machine learning module on the cloud to process multimodal data to detect health state and predict health deterioration of the wearer.

### Method of Use

In one aspect, the present invention provides a method of monitoring a physiological or environmental data. For example, in one embodiment, the method comprises the use of a smart wearable IOT device as described in this application to monitor one or more of a subject's biometrics or environmental data of the subject's surroundings.

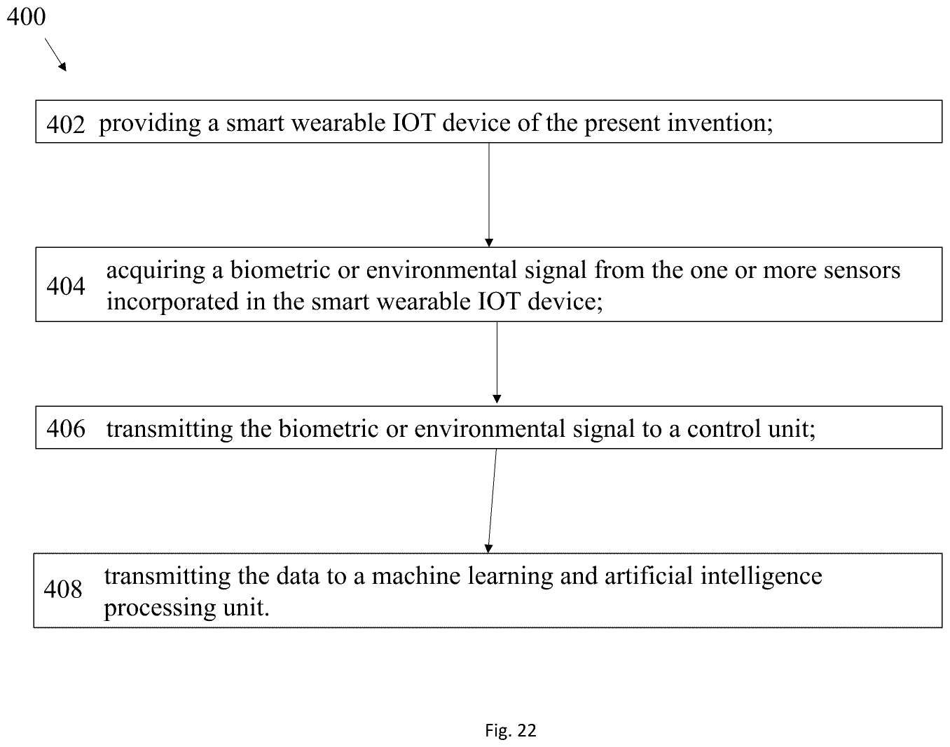

Referring now to FIG. 22, an exemplary method 400 of using a smart wearable IOT device is depicted. Method 400 begins with step 402, wherein the smart wearable IOT device as described above is provided. In step 404, wearable IOT device is positioned on a subject. In step 406, a biometric or environmental signal from the one or more sensors is acquired. In one embodiment, the biometric signals correspond to biological or physiological characteristics of the subject. In one embodiment, the environmental signal corresponds to the environmental data of the surroundings of the subject. In one embodiment, the environmental signal may correspond to the distance of the wearer with another wearer or other objects. In step 406, the biometric or environmental signal or both is transmitted to a control unit. In one embodiment, the control unit may interpret the data received from the sensors. In some embodiments, the control unit may make one or more comparisons of the data computed with the threshold value stored in memory. It will be apparent to persons of ordinary skill in the art that the interpretation of raw data may comprise other analysis of the biological data that will characterize a physiological condition of the body. In step 408, the control unit transmits the data to a machine learning and artificial intelligence processing unit. In one embodiment, the machine learning and artificial intelligence processing unit is adapted to receive and process the data and the contextual data. In one embodiment, the machine learning and artificial intelligence processing unit is adapted to identify one or more patterns of the subject's body based on processing of the data and the contextual data. In one embodiment, the machine learning and artificial intelligence processing unit is adapted to compare the patterns of the subject's body, and to determine risks related to the diagnostic condition of the subject. In one embodiment the machine learning system can predict the upcoming adverse events and evolution of infection.

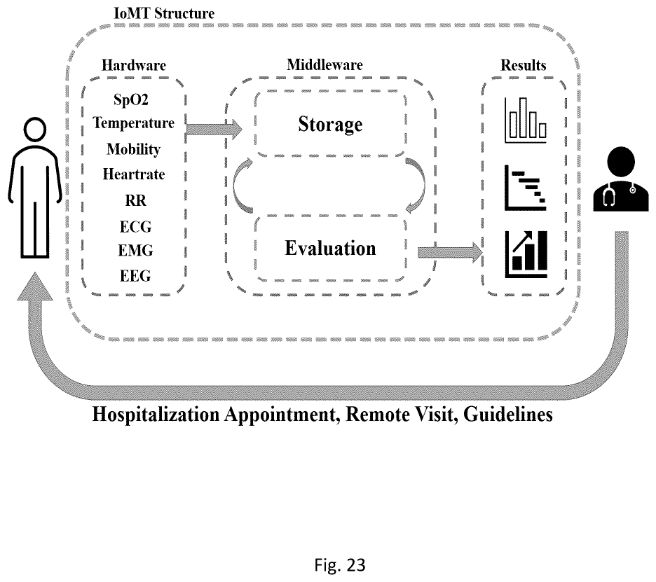

In one embodiment, the methods are used for personal use (see FIG. 23) to monitor biometric and/or environmental signals, store and evaluate signals. For example, the method can be used to predict the risk of infection, diagnose infection, and the like. In one embodiment, the methods are used to trigger additional intervention such as hospitalization, remote or in-person evaluation, quarantine, and the like.

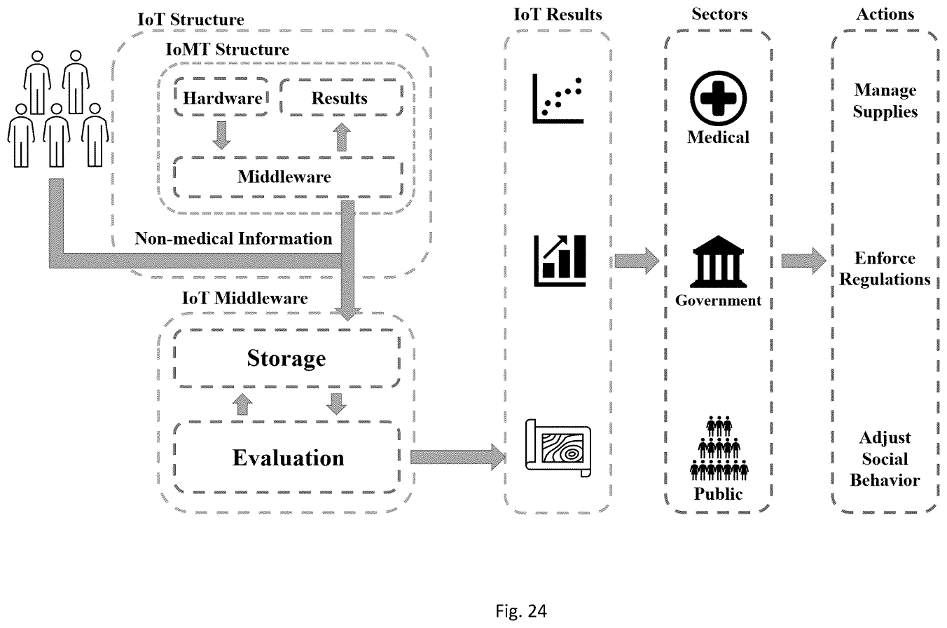

In one embodiment, the methods are used in a population, such as a population of high-risk subjects (see FIG. 24), to monitor the biometric and/or environmental signals from individuals within the population. In one embodiment, the methods can be used to determine or detect the close contacts of an individual. In certain embodiments, the methods can be used to determine the spatial distance between individuals or between an individual and an external beacon. In one embodiment, the methods can be used to determine the duration in which individuals were within a defined space or were within a defined distance from another individual. In certain aspects, the methods can be used to gather crowd data to be used by various sectors, including but not limited to a hospital unit, a residential care unit, a physical therapy unit, a gym, the medical community, government, employers, and the like.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A smart wearable IOT neckpiece device comprising:
a sensing module comprising a front side, a rear side, a top side, a bottom side, a central part, a first end and a second end, wherein the front side comprises at least one opening and wherein the sensing module comprises a charging probe;
a shaft having a proximal end, a distal end and a lumen therebetween, wherein the distal end is positioned in the central part and the proximal end is positioned within the at least one opening;
at least one component comprising a first opening and a second opening, wherein the second opening is in fluid communication with the proximal end and wherein the first opening is configured to have a larger diameter than the second opening;
at least one microphone;
a transmit circuit; and
a power source.

2. The smart wearable IOT neckpiece device of claim 1, wherein the sensing module has an asymmetric front side and rear side, and wherein the front side is concave inwards with respect to a horizontal plane, and the rear side is convex outward with respect to the horizontal plane.

3. The smart wearable IOT neckpiece device of claim 2, wherein the front side is configured to follow along the curvature of a subject's neck.

4. The smart wearable IOT neckpiece device of claim 1, wherein the first opening is configured to contact a subject's skin.

5. The smart wearable IOT neckpiece device of claim 1, wherein the at least one microphone is positioned in distal end of the shaft.

6. The smart wearable IOT neckpiece device of claim 1, wherein the at least one microphone is positioned in distal end of the shaft and at least one microphone is positioned within the at least one opening for stereo configuration to configure a differential recording for ambient noise rejection.

7. The smart wearable IOT neckpiece device of claim 1, wherein the transmit circuit is positioned in the central part and is configured to transmit digital waveforms received from the at least one microphone to one selected from the group consisting of a remote computer, a smart phone, a handheld personal digital assistant (PDA) device and combinations thereof.

8. The smart wearable IOT neckpiece device of claim 1, wherein the transmit circuit comprises one selected from the group consisting of a WiFi module or a Bluetooth module or combinations thereof.

9. The smart wearable IOT neckpiece device of claim 1, wherein the power source is a rechargeable battery.

10. The smart wearable IOT neckpiece device of claim 1, wherein the charging probes are positioned on the bottom side and have magnetic interfaces.

11. The smart wearable IOT neckpiece device of claim 1, wherein the device further comprises a charging station, wherein the charging station comprises a housing unit having a cavity configured to conform to the shape of the sensing module.

12. The smart wearable IOT neckpiece device of claim 11, wherein the cavity comprises magnets strategically oriented and aligned with the charging probes, so as to force the sensing module to self-align with the charging station.

13. The smart wearable IOT neckpiece device of claim 1, wherein the device further comprises a charging station, wherein the charging station comprises a battery that connects to the sensing module through a wired connection for charging.

14. The smart wearable IOT neckpiece device of claim 1, wherein the device further comprises a securement member.

15. The smart wearable IOT neckpiece device of claim 14, wherein the securement member is selected from the group consisting of a Velcro strap, elastic strap, headband or a double sided connection patch.

16. The smart wearable IOT neckpiece device of claim 1, wherein the securement member comprises an adhesive to secure the device to the skin.

17. A smart monitoring headpiece device comprising:
at least one sensor module comprising one or more sensors, a battery, a wireless transceiver, a charging circuit, a data recording module, and a control unit; and
a securement member.

18. The smart monitoring headpiece device of claim 17, wherein the sensor module is positioned within a housing unit.

19. The smart monitoring headpiece device of claim 17, wherein the housing unit comprises a flexure mechanical auto-adaptation feature that adapts to different sizes and shapes of foreheads of users.

20. The smart monitoring headpiece device of claim 17, wherein the one or more sensor is selected from the group consisting of a temperature sensor, a presence sensor, a global positioning sensor, accelerometer sensor, IMU, one or two acoustic sensor, a gyroscope sensor, a magnetic sensor, a distance sensor, a skin contact pressure sensor, SpO2 sensor, heartrate sensor, optical Plethysmography (PPG) sensor, an ECG sensor, a respiratory rate sensor, a microphone, cough detection sensor, mobility sensor, step count sensor, Bluetooth module, Bluetooth low energy sensor (and/or WiFi) for detecting social distancing between different wearer and for data transmission, and combinations thereof.

21. The smart monitoring headpiece device of claim 17, wherein the one or more sensors, battery, data recording module and charging circuit, are housed outside the sensor module in an external unit.

22. The smart monitoring headpiece device of claim 17, wherein the housing unit comprises a main body and two side wings, and wherein the two side wings are configured to flex and allow the headpiece device directly contact or remain proximal to skin portion of a subject's head.

23. The smart monitoring headpiece device of claim 23, wherein the main body holds one or more sensors that are to be separated by a distance from the skin of the user, and wherein one or more of the side wings comprise one or more sensors that are to be in contact with the skin of the user.

24. The smart monitoring headpiece device of claim 17, wherein the device further comprises a data recording module configured to save data on a removable memory card.

25. The smart monitoring headpiece device of claim 18, wherein the battery is configured to charge the sensor module and one or more sensors in the housing unit.

26. A system comprising:
a) a neckpiece device comprising:
a sensing module comprising a front side, a rear side, a top side, a bottom side, a central part, a first end and a second end, wherein the front side comprises at least one opening and wherein the sensing module comprises a charging probe;
a shaft having a proximal end, a distal end and a lumen therebetween, wherein the distal end is positioned in the central part and the proximal end is positioned within the at least one opening;
at least one component comprising a first opening and a second opening, wherein the second opening is in fluid communication with the proximal end and wherein the first opening is configured to have a larger diameter than the second opening;
at least one microphone;
a transmit circuit;
a power source; and
b) a headpiece device comprising:
a sensor module comprising one or more sensors, a battery, a wireless transceiver, a charging circuit, a data recording module, and a control unit; and
a securement member.
